(19) United States
(12) Patent Application Publication
(10) Pub. No.: US 2017/0266252 A1
(43) Pub. Date: Sep. 21, 2017

(54) GLYCINE MAX CONSTRUCTS, SOY PROTEIN SEQUENCES, AND METHODS OF TREATING HEALTH CONDITIONS USING THE SAME

(71) Applicant: Kieu Hoang, Agoura Hills, CA (US)

(72) Inventor: Kieu Hoang, Agoura Hills, CA (US)

(21) Appl. No.: 14/979,697

(22) Filed: Dec. 28, 2015

Related U.S. Application Data
(60) Provisional application No. 62/096,832, filed on Dec. 24, 2014.
(60) Provisional application No. 62/102,123, filed on Jan. 12, 2015.

(51) Int. Cl. A61K 38/02; A23L 11/00; A23L 33/105; A61K 36/48; A23L 33/00

(52) U.S. Cl. CPC A61K 38/02; A61K 36/48; A23L 33/30; A23L 33/105; A23L 11/03; A23V 2002/00

(57) ABSTRACT

The present subject matter is directed to a food composition for human consumption comprising a plurality of soy ingredients, wherein the plurality of soy ingredients comprise flesh and skin from soy, and wherein the plurality of soy ingredients further comprise a soy construct comprising one or more of 359 soy proteins found in immature soy (KH 111) and mature soy (KH 103). The present subject matter is directed to a method of treating certain diseases in a patient comprising administering the food composition to a patient in need thereof. The present subject matter is further directed to a method of preparing a soy construct by processing Glycine max plants.

### RELATED APPLICATIONS

The present patent application claims priority to provisional U.S. Patent Application No. 62/096,832 filed Dec. 24, 2014 and provisional U.S. Patent Application No. 62/102,123 filed Jan. 12, 2015, which were filed by the inventor hereof and are incorporated by reference herein in their entirety.

### TECHNICAL FIELD

The present subject matter relates to Glycine max constructs and soy protein sequences. In particular, the present subject matter is associated with methods of treating health conditions using the glycine max constructs and soy protein sequences.

### BACKGROUND

The soybean plant (Glycine max) is popular for its use in food products because of its favorable health implications when consumed by humans. There are many well-known soy extraction processes. However, they typically do not make use of the entire plant, including the skin, which has useful components that can be healthfully consumed by humans. It is therefore desirable to develop additional soy extraction processes that make use of a greater percentage of the plants as a whole and reduce the waste involved with such processes.

### SUMMARY

An embodiment of the present subject matter is directed to a food composition for human consumption comprising a plurality of soy ingredients, wherein the plurality of soy ingredients comprise flesh and skin from soy, wherein the plurality of soy ingredients further comprise a soy construct comprising one or more of 359 soy proteins found in immature soy (KH 111) and mature soy (KH 103).

An embodiment of the present subject matter is directed to a food composition for human consumption comprising a plurality of soy ingredients, wherein the plurality of soy ingredients comprise flesh and skin from soy, wherein the plurality of soy ingredients further comprise a soy construct comprising one or more of 359 soy proteins found in immature soy (KH 111) and mature soy (KH 103), wherein the plurality of soy ingredients comprises a combination of at least two of 359 proteins synthesized by particular Soy KH healthy cells, wherein the Soy KH healthy cells send signals to damaged or sick cells, thereby triggering synthesis of proteins to transform the damaged or sick cells to become healthy, wherein the Soy KH healthy cells send signals to other undamaged cells to synthesize proteins to protect the other undamaged cells from damaged, infected, and prone to DNA and other cellular alterations, and wherein the Soy KH healthy cells send signals to a body to produce new cells that are healthy, thereby preventing the new cells from being affected by intracellular and extracellular damaging signals.

An embodiment of the present subject matter is directed to a food composition for human consumption comprising a plurality of soy ingredients, wherein the plurality of soy ingredients comprise flesh and skin from soy, wherein the plurality of soy ingredients further comprise a soy construct comprising one or more of 359 soy proteins found in immature soy (KH 111) and mature soy (KH 103), wherein the plurality of soy ingredients comprises a single protein from the 359 proteins synthesized by particular Soy KH healthy cells, wherein the Soy KH healthy cells send signals to damaged or sick cells, thereby triggering synthesis of proteins to transform the damaged or sick cells to become healthy, wherein the Soy KH healthy cells send signals to other undamaged cells to synthesize proteins to protect the other undamaged cells from being damaged, infected, and prone to DNA and other cellular alterations, and wherein the Soy KH healthy cells send signals to a body to produce new cells that are healthy, thereby preventing the new cells from being affected by intracellular and extracellular damaging signals.

An embodiment of the present subject matter is directed to a method of preparing a soy construct comprising processing Glycine max plants immediately after harvest; washing the Glycine max plants; grinding the Glycine max plants and collecting raw juice; testing pH of the raw juice; centrifuging the raw juice and collecting a first precipitate and a first supernatant; adding water to the first precipitate to form a water-precipitate solution, mixing the water-precipitate solution, and centrifuging the water-precipitate solution, and collecting a second precipitate and a second supernatant from the water-precipitate solution; mixing the first supernatant, testing pH of the first supernatant, centrifuging the first supernatant, and collecting supernatant juice; and collecting all precipitate, combining, and capsulizing the combined precipitate.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1 is a flow chart depicting methods of processing Glycine max into purified soy constructs including albumin-like, APOA1-like, juice products, powder products, and soy precipitate capsules.

FIG. 2 is a graph depicting the cell count of soy powder from mature soy processed according to embodiments of the present subject matter (KUNAKIN™) after dilution with WFI at various ratios—the initial dilution being 1:10 (shown on the X-axis as dilution 1). The graph includes cell counts for powder to WFI dilution ratios of 1:10, 1:25, 1:50, 1:100, 1:200, and 1:400.

FIG. 3 is a graph depicting the cell count of unprocessed mature soy (KH 103) after dilution with WFI at various ratios—the initial dilution being 1:10 (shown on the X-axis as dilution 1). The graph includes cell counts for mature soy to WFI dilution ratios of 1:10, 1:25, 1:50, 1:100, and 1:200.

FIG. 4 depicts a SDS-PAGE analysis of mature soy concentrate and permeate compared with human albumin (HA), intravenous immunoglobulin (IVIG), and apolipoprotein A-I (APOA1).

FIG. 5 depicts a SDS-PAGE analysis of mature soy and mature soy concentrate.

FIG. 6 depicts a graphical representation of serum protein electrophoresis of soy powder from mature soy processed according to embodiments of the present subject matter.

FIG. 7 is a table comparing TC, HDL, LDL/VLDL, and TG levels for various plants, foods, and beverages.

FIG. 8 depicts the quantification levels of TC, HDL, LDL/VLDL, and TG for mature soy (KH 103) and immature soy (KH 111).

FIG. 9 is a graphical representation of median relative bioluminescence vs days after leukemia cell injection for animals treated with mature and immature soy constructs.

FIGS. 10A, 10B & 10C are images of animals treated prophylactically with mature soy constructs vs vehicle and positive control groups.

FIGS. 11A, 11B & 11C are images of animals treated therapeutically with mature soy constructs vs vehicle and positive control groups.

FIGS. 12A, 12B & 12C are images of animals treated prophylactically with immature soy constructs vs vehicle and positive control groups.

FIGS. 13A, 13B & 13C are images of animals treated therapeutically with immature soy constructs vs vehicle and positive control groups.

FIG. 14 is an image of SDS Page #1 for soy protein.

FIG. 15 is an image of SDS Page #2 for soy protein KH901.

FIG. 16 is an image of SDS Page #3a for supernatant of soybean.

FIG. 17 is an image of SDS Page #3b for paste of soybean.

FIG. 18 is a diagram of the present subject matter for preventing, curing, and protecting against disease.

FIG. 19 is an image of RAAS KUNAKIN™ inhibiting growth of lung cancer cells, where the count of KH good healthy cells is 7,050,000/ml.

FIG. 20 is an image of RAAS Wine inhibiting lung cancer cells.

FIG. 21 is an image of RAAS Wine inhibiting breast cancer cells.

FIG. 22 is an image of RAAS Nutrionals in vivo testing of leukemia, particularly an outline of studies conducted in mice.

FIG. 23 is an image of RAAS Nutrionals in vivo testing of leukemia, particularly RAAS supplements preventing the development and progression of leukemia.

FIG. 24 is an image of RAAS Nutrionals in vivo testing of leukemia.

FIG. 25 is an image of RAAS Nutrionals in vivo testing of leukemia, particularly tumor-free survival of animals treated with KHJ or KH103 and overall survival of animals treated with KHL or KH103.

FIG. 26 is an image of RAAS Nutrionals in vivo testing of leukemia, particularly RAAS nutrionals in prophylactic and therapeutic treatment on murine leukemia model.

FIG. 27 is an image of RAAS Nutrionals in vivo testing of breast cancer, particularly the study design to test efficacy of RAAS supplements against breast cancer in mice model.

FIG. 28 is an image of RAAS Nutrionals in vivo testing of breast cancer, particularly MBA-MB-436 efficacy results of prophylactic groups and tumor volume and body weight change.

FIG. 29 is an image of RAAS Nutrionals in vivo testing of breast cancer, particularly MDA-MB-436 efficacy results of therapeutic groups and tumor volume and body weight change.

FIG. 30 is an image of RAAS Nutrionals in vivo testing of breast cancer, particularly leukocyte count and tumor volume at D0 and D51 post the start of treatment.

FIG. 31 is an image of RAAS Nutrionals in vitro testing of diabetics, particularly RAAS supplements stimulating glucose uptake in 3T3-L1 adipocytes.

FIG. 32 is an image of RAAS Nutrionals in vitro testing of diabetics, particularly lipids content in RAAS Nutrionals products.

FIG. 33 is an image of RAAS Nutrionals in vitro testing of diabetics, particularly study design to analyze the effectiveness of RAAS supplements on diabetics using DIO mice model.

FIG. 34 is an image of RAAS Nutrionals in vitro testing of diabetics, particularly blood glucose levels and AUC (0-8 weeks).

FIG. 35 is an image of RAAS Nutrionals in vitro testing of diabetics, particularly OGTT and OGTT glucose AUC (0-120 min).

FIG. 36 is an image of RAAS Nutrionals in vitro testing of diabetics, particularly insulin levels and AUC (0-8 weeks).

FIG. 37 is an image of RAAS Nutrionals in vitro testing of diabetics, particularly plasma TG levels and AUC (0-8 weeks).

FIG. 38 is an image of RAAS Nutrionals in vitro testing of diabetics, particularly plasma TCHO levels and AUC (0-8 weeks).

FIG. 39 is an image of RAAS Nutrionals in vitro testing of diabetics, particularly plasma HDL levels and AUC (0-8 weeks).

FIG. 40 is an image of RAAS Nutrionals in vitro testing of diabetics, particularly plasma LDL levels and AUC (0-8 weeks).

FIG. 41 is an image of RAAS Nutrionals in vitro testing of anti-viral activity, particularly neutralization of HIV-1 Env-psuedotyped.

FIG. 42 is an image of RAAS Nutrionals in vitro testing of anti-viral activity, particularly anti-HIV activity of human plasma derive proteins on HIV-RT Enzyme.

FIG. 43 is an image of RAAS Nutrionals in vitro testing of anti-viral activity, particularly RAAS products against HCV genotype 1a, 1b and 2a replicons for antiviral activity.

FIG. 44 is an image of RAAS Nutrionals in vitro testing of anti-viral activity, particularly RAAS products displayed potent antival activity against influenza virus.

FIG. 45 is an image of RAAS Nutrionals in vitro testing of anti-viral activity, particularly the effect of prophylactic treatment or therapeutic treatment of RAAS 8 or ETV inmouse.

FIG. 46 is an image of antiviral activity of RAAS Products against HBV in vivo, particularly the effect of RAAS 8 or ETV on the intermediate HBV replication in the mouse livers.

FIG. 47 is an image of antiviral activity of RAAS Products aginst influenza in vivo, particularly the effect of AFOD RAAS2 on H1N1-caused mouse mortality.

FIG. 48 is a chart directed to testimonial kidney function.

FIG. 49 is a chart directed to testimonial diabetes.

FIG. 50 is a chart directed to testimonial cardiovascular disease.

FIG. 51 is a table directed to testimonial blood lipids level.

FIG. 52 is an image directed to family members and relatives with medical conditions.

FIG. 53 is a chart directed to hypocholesterolemic and hypoglycemic effects for friends.

FIG. 54 is a chart directed to hypocholesterolemic and hypoglycemic effects for relatives.

FIGS. 55A-G are charts directed to hypocholesterolemic and hypoglycemic effects for my family.

FIG. 56 is an image directed to the effects of APOA-1 in removing fats in rabbit.

FIG. 57 is a chart directed to types of pain and corresponding tests and treatments.

FIG. 58 is an image directed to coloration and separation of a product.

FIG. 59 is a chart directed to summary of the hypoglycemic effect from all personal observations.

FIG. 60 is an image directed to a testimonial from drinking KIEU HOANG™ wine.

FIG. 61 is a chart directed to the hypoglycemic effects in mice.

FIG. 62 is an image directed to a testimonial for inflammation in KIEU HOANG™.

FIG. 63 is an image directed to a testimonial for inflammation, particularly mesenteric adenitis in a 4-year-old male.

FIG. 64 is an image directed to a testimonial for gall bladder stones in a 40-year-old male.

FIG. 65 is a chart and image directed to a testimonial for a friend diagnosed with chronic lymphocytic leukemia.

FIG. 66 is an image directed to a testimonial for lung cancer.

FIG. 67 is an image directed to a testimonial for lung cancer.

FIG. 68 is a series of images directed to a testimonial for myeloma.

FIG. 69 is an image directed to a testimonial for myeloma.

FIG. 70 is an image directed to a testimonial for liver cancer.

FIG. 71 is an image directed to a testimonial for breast DCIS and opinions from various institutes.

FIG. 72 is a series of MRI mass measurement images directed to a testimonial for breast DCIS.

FIG. 73 is an image directed to breast cancer cells in a testimonial.

FIG. 74 is a series of images directed to Sundia Recovered 4-6: re-implanted and immune cell (immune deficiency mice).

FIG. 75 is a chart directed to mice vs. human living ages after recovery from leukemia.

FIG. 76 is an image directed to a testimonial for hepatitis B virus.

FIG. 77 is a chart directed to RAAS Products suppressing HVB replication and killing hepatitis B virus in mice.

FIG. 78 is an image directed to a testimonial for hepatitis C virus.

FIG. 79 is a series of images directed to H&E staining for G9 of the livers in KHGD treated mice-therapeutic (20×) where wine did not damage the livers.

FIG. 80 is a chart directed to AFOD saved rats from the lethod model of Parkinson's disease.

FIG. 81 is a series of images directed to a Parkinson's disease Model where Parkinson was induced with 6-OHDA injection.

FIG. 82 is an image directed to a testimonial for vertigo, dizziness, headache, fear of cold weather, eyes blurry, and ear ringing.

FIG. 83 is an image directed to a testimonial for vertigo, dizziness, headache, fear of cold weather, eyes blurry, and ear ringing.

FIGS. 84A, 84B & 84C are images directed to a plants protein wherein the sequenced proteins have the same molecular weight as human immunoglobulin and APOA-1.

FIG. 85 is a chart directed to RAAS Nutritionals supplements containing more than 350 proteins in each capsule.

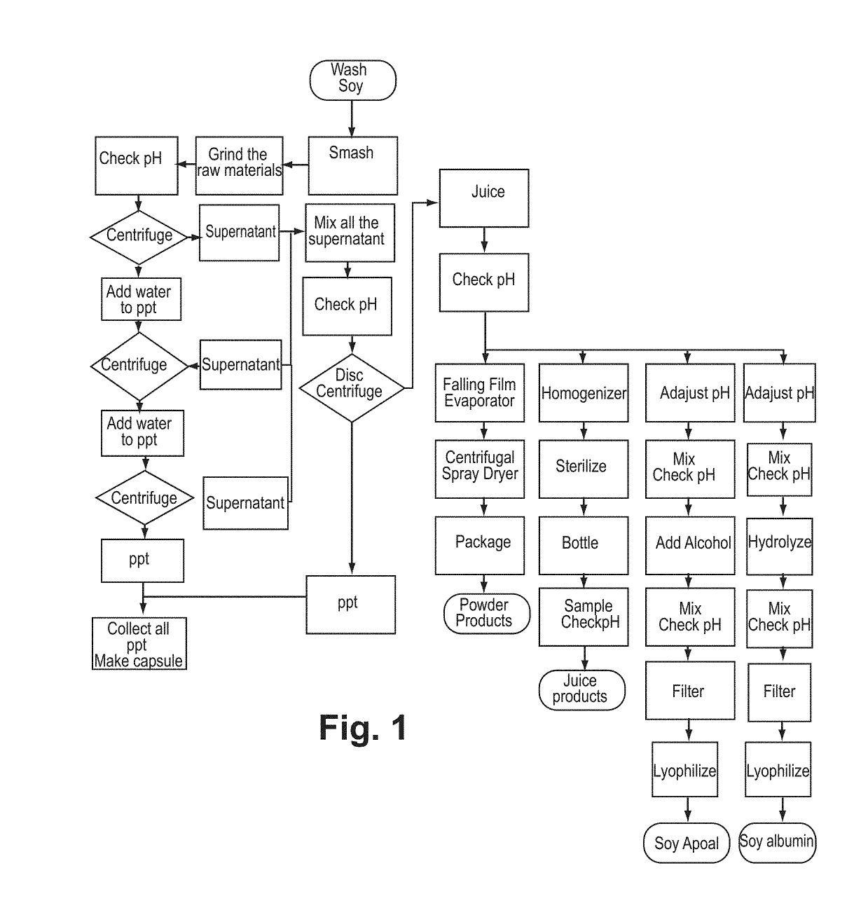

### DETAILED DESCRIPTION

Unless defined otherwise all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language; however, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

The present subject matter is directed to a process for producing soy compositions for use in beverages, purified juice, proteins, and powder derived from Glycine max. The protein makeups of the compositions and amino acid sequences of the same have been analyzed and are found to contain 134 previously undiscovered proteins and 225 previously discovered proteins. Embodiments of the present subject matter include soy constructs containing one or more of the 359 previously undiscovered and previously discovered soy proteins found in immature and mature soy, as well as methods of treating various health conditions such as leukemia and atherosclerosis through consumption or administration of those constructs.

According to the present subject matter, soy constructs manufactured from immature and mature soy containing various combinations of previously undiscovered and previously discovered soy proteins are utilized in the prophylactic and therapeutic treatment of leukemia and other health conditions. Soy products, including daily dietary supplements, containing one or more of these 359 proteins assist in lowering triglyceride and cholesterol levels while increasing HDL High Density Lipoprotein (APOA1) levels. This shift lowers the buildup of atherosclerosis and the incidence of stroke. Other applications of these formulations are currently being investigated including the inhibition and prevention of diabetes.

The present subject matter describes processes for the production of purified soy juice, powder, and proteins, as well as compositions formed thereby. Certain preferred, non-limiting, embodiments and means for producing soy products containing 134 previously undiscovered proteins and 225 previously discovered proteins from Glycine max are set forth in detail herein.

An embodiment of the present subject matter is directed to a food composition for human consumption comprising a plurality of soy ingredients, wherein the plurality of soy ingredients comprise flesh and skin from soy, wherein the plurality of soy ingredients further comprise a soy construct comprising one or more of 359 soy proteins found in immature soy (KH 111) and mature soy (KH 103).

An embodiment of the present subject matter is directed to a food composition, wherein a KH 103 formulation comprises 359 cells synthesized from 359 proteins, wherein the formulation comprises Soy KH healthy cells, wherein the formulation further comprises 225 previously found proteins and 134 newfound proteins and cells in which RNA synthesizes good proteins.

In an embodiment, a temperature during manufacturing is −10° C.-250° C.

In an embodiment, pH varies from 2.5-10.

In an embodiment, a food composition comprises bacteria and virus inactivation using a combination of pasteurization and dry high temperature heating.

In an embodiment, a KH 103 formulation prevents development of leukemia in mice after two times of implantation with a total of 30,000,000 Leukemia cancer cells through vein injection. In an embodiment, the first implantation is with 10,000,000 cells and the second implantation is with 20,000,000 cells for mice that did not develop leukemia after the first implantation.

In an embodiment, the food composition is a powder or a juice. In an embodiment, the powder is a mixed food supplement. In an embodiment, the powder or juice comprises at least 359 proteins. In an embodiment, the powder or juice comprises 134 proteins. In an embodiment, the powder or juice comprises 134 proteins synthesized by 134 cells.

In an embodiment, the composition comprises Albumin from said KH 103 formulation having a molecular weight similar to Human Albumin.

In an embodiment, the composition comprises APOA1 (High Density Lipoprotein) from said KH 103 formulation having a molecular weight similar to Human APOA1 (High Density Lipoprotein).

In an embodiment, the composition comprises Alpha 1 Antitrypsin from said KH 103 formulation having a molecular weight similar to Human Alpha 1 Antitrypsin.

In an embodiment, the composition comprises Alpha 1, Alpha 2, Beta, and Gamma proteins from said KH 103 formulation having a molecular weight similar to Human Immune Globulins.

An embodiment of the present subject matter is directed to a method of treating certain diseases in a patient comprising administering the food composition to a patient in need thereof, wherein the food composition has a concentration of soy proteins found in KH 103 above 0%.

In an embodiment, administration of the food composition lowers triglycerides and cholesterol and increases High Density Lipoprotein (APOA1) in the patient.

In an embodiment, administration of the food composition cleans plaque and provides heart, brain, and artery blockage protection in the patient.

In an embodiment, administration of the food composition inhibits growth of various cancer cells in the patient.

In an embodiment, administration of the food composition suppresses inflammation in the patient due to different causes of disease.

An embodiment of the present subject matter is directed to a food composition for human consumption comprising a plurality of soy ingredients, wherein the plurality of soy ingredients comprise flesh and skin from soy, wherein the plurality of soy ingredients further comprise a soy construct comprising one or more of 359 soy proteins found in immature soy (KH 111) and mature soy (KH 103), wherein the plurality of soy ingredients comprises a combination of at least two of 359 proteins synthesized by particular Soy KH healthy cells, wherein the Soy KH healthy cells send signals to damaged or sick cells, thereby triggering synthesis of proteins to transform the damaged or sick cells to become healthy, wherein the Soy KH healthy cells send signals to other undamaged cells to synthesize proteins to protect the other undamaged cells from damaged, infected, and prone to DNA and other cellular alterations, and wherein the Soy KH healthy cells send signals to a body to produce new cells that are healthy, thereby preventing the new cells from being affected by intracellular and extracellular damaging signals.

An embodiment of the present subject matter is directed to a food composition for human consumption comprising a plurality of soy ingredients, wherein the plurality of soy ingredients comprise flesh and skin from soy, wherein the plurality of soy ingredients further comprise a soy construct comprising one or more of 359 soy proteins found in immature soy (KH 111) and mature soy (KH 103), wherein the plurality of soy ingredients comprises a single protein from the 359 proteins synthesized by particular Soy KH healthy cells, wherein the Soy KH healthy cells send signals to damaged or sick cells, thereby triggering synthesis of proteins to transform the damaged or sick cells to become healthy, wherein the Soy KH healthy cells send signals to other undamaged cells to synthesize proteins to protect the other undamaged cells from being damaged, infected, and prone to DNA and other cellular alterations, and wherein the Soy KH healthy cells send signals to a body to produce new cells that are healthy, thereby preventing the new cells from being affected by intracellular and extracellular damaging signals.

An embodiment of the present subject matter is directed to a method of preparing a soy construct comprising processing Glycine max plants immediately after harvest; washing the Glycine max plants; grinding the Glycine max plants and collecting raw juice; testing pH of the raw juice; centrifuging the raw juice and collecting a first precipitate and a first supernatant; adding water to the first precipitate to form a water-precipitate solution, mixing the water-precipitate solution, and centrifuging the water-precipitate solution, and collecting a second precipitate and a second supernatant from the water-precipitate solution; mixing the first supernatant, testing pH of the first supernatant, centrifuging the first supernatant, and collecting supernatant juice; and collecting all precipitate, combining, and capsulizing the combined precipitate.

In an embodiment, a final juice product is produced by homogenizing the supernatant juice; sterilizing the supernatant juice; bottling the supernatant juice; and checking pH of the supernatant juice.

In an embodiment, a final powder product is produced by concentrating the supernatant juice to create a concentrated juice; spray drying the concentrated juice to produce a powder; and packaging the powder.

In an embodiment, a final APOA1 extract product is produced by adjusting pH of the supernatant juice and mixing; adding alcohol to the supernatant juice to create a juice-alcohol solution and mixing; filtering the juice-alcohol solution to create a filtered solution; and lyophilizing and packaging the filtered solution.

In an embodiment, a final albumin extract product is produced by adjusting pH of the supernatant juice and mixing; hydrolyzing the supernatant juice and mixing to create a hydrolyzed juice; filtering the hydrolyzed juice to create a filtered juice; and lyophilizing and packaging the filtered juice.

### EXAMPLES

According to an embodiment of the present subject matter, Glycine max is processed immediately after harvest. The plants are washed in a cleaning bath at about 0-100° C. The whole plants are processed in a grinder to cut the plants into small particles at about 0-100° C. The raw materials are then collected. The raw materials are ground in a superfine mill at about 0-100° C. The raw juice is then collected. The pH of the raw juice is tested and should preferably be in the range of about 2.5-10. The raw juice is centrifuged at low to high rpm with a normal centrifuge at about −30-30° C. The precipitate and supernatant are then collected.

In an embodiment, two to four times the volume of the precipitate of water is added to the precipitate. The water-precipitate solution is mixed for approximately 10-100 minutes and centrifuged again at low to high rpm with a normal centrifuge at about −30-60° C. The precipitate and supernatant are then collected.

In an embodiment, one to two times the volume of the precipitate of water is added to the precipitate. The water-precipitate solution is mixed for approximately 10-100 minutes and centrifuged again at low to high rpm with a normal centrifuge at about −30-60° C. The precipitate and supernatant are then collected.

In an embodiment, the supernatant is mixed for about 10-100 minutes at about 0-100° C. The pH of the mixed supernatant is tested and should preferably be in the range of about 2.5 to 10. The supernatant juice is centrifuged at moderate to high rpm with a disc centrifuge at about −30-60° C. and the juice is collected. The pH of the juice is tested and should preferably be in the range of about 2.5 to 10.

### Juice Product

In an embodiment, a final juice product is obtained by the following. The juice is homogenized at 40-60 MPa. The juice is then sterilized at about 100-180° C. for 5-20 seconds with a pipe sterilizer. The clear juice is bottled at 25-35° C. The pH of the bottled juice is then checked, which should preferably be in a range of about 3.8-7.6.

### Powder Product

In an embodiment, a final powder product is obtained by the following. The juice is concentrated in a single-effect falling film evaporator at about 20-100° C. until the brix value is 20-100. The concentrated juice is transferred to a centrifugal spray dryer in which the powder is produced. The powder is then packaged.

### APOA1 Extract Product

In an embodiment, a final APOA1 extract product is obtained by the following. The pH of the juice is adjusted to 3-10 at a low temperature. The juice is mixed at a low temperature and the pH of 3-10 is maintained. Alcohol is added to the juice at low temperature. The juice-alcohol solution is mixed at a low temperature and the pH of 3-10 is maintained. The mixed juice-alcohol solution is filtered at low temperature. The filtered solution is lyophilized and packaged.

### Albumin Extract Product

In an embodiment, a final albumin extract is obtained by the following. The pH of the juice is adjusted to 3-10 at about 20-23° C. The juice is mixed at about 20-23° C. while maintaining a pH of 3-10. The juice is hydrolyzed at 20-80° C. The hydrolyzed juice is mixed while maintaining a pH of 3-10. The juice is filtered at low temperature. The filtered juice is lyophilized and packaged.

### Capsule Product

In an embodiment, all of the precipitate is mixed and capsulized to obtain a final capsule product.

### In Vitro Examination: Cell Count Analysis of Soy Powder

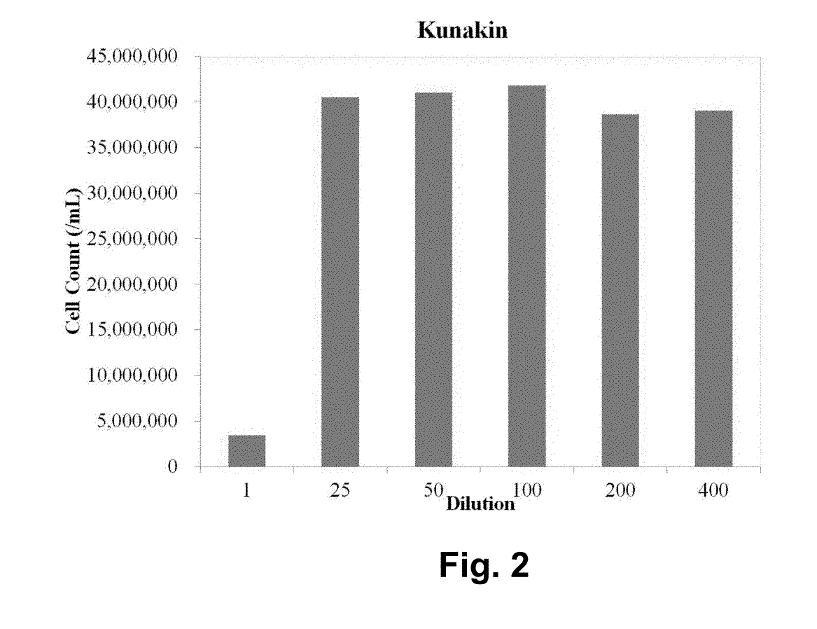

Soy powder processed from mature soy plants according to embodiments of the present subject matter was diluted with WFI at a ratio of 1:10 as depicted in FIG. 2 as dilution 1 and a cell count was taken. Further cell counts were taken for dilution ratios of 1:25, 1:50, 1:100, 1:200, and 1:400. Cell counts for this product were found to range from approximately one billion to sixteen billion cells per mL.

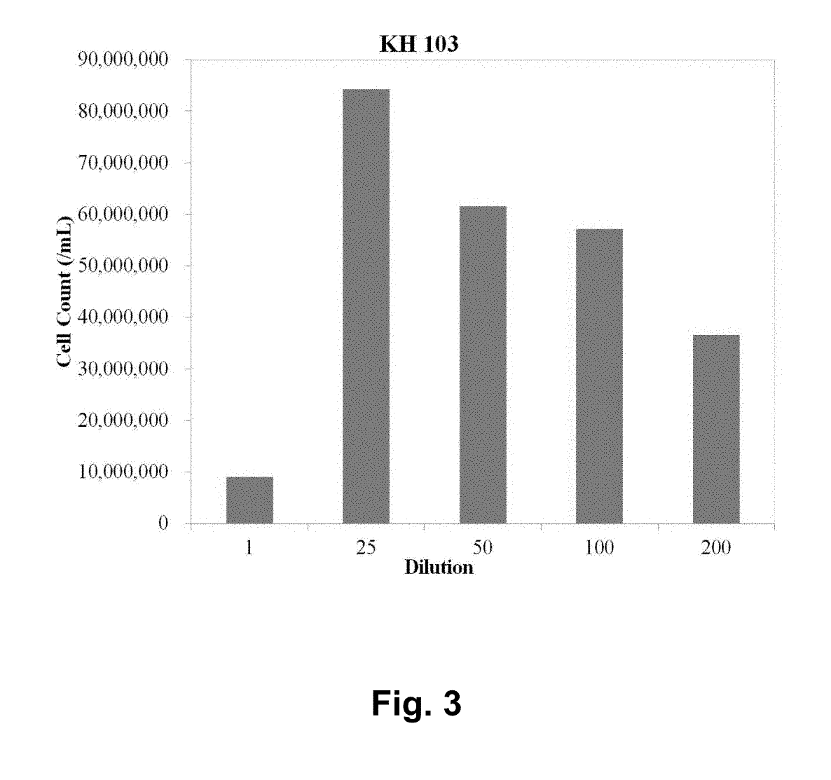

The cell count of mature soy plants at various WFI dilution ratios was also analyzed as depicted in FIG. 3. Cell counts for this product were found to range from approximately two billion to eight billion cells.

### In Vitro Examination: Sds-Page Analysis of Glycine max

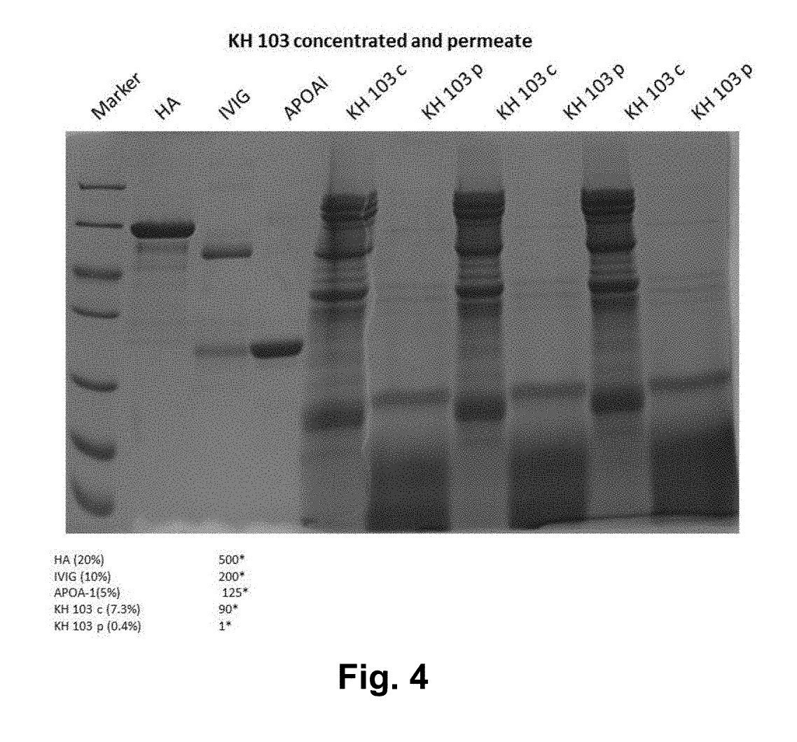

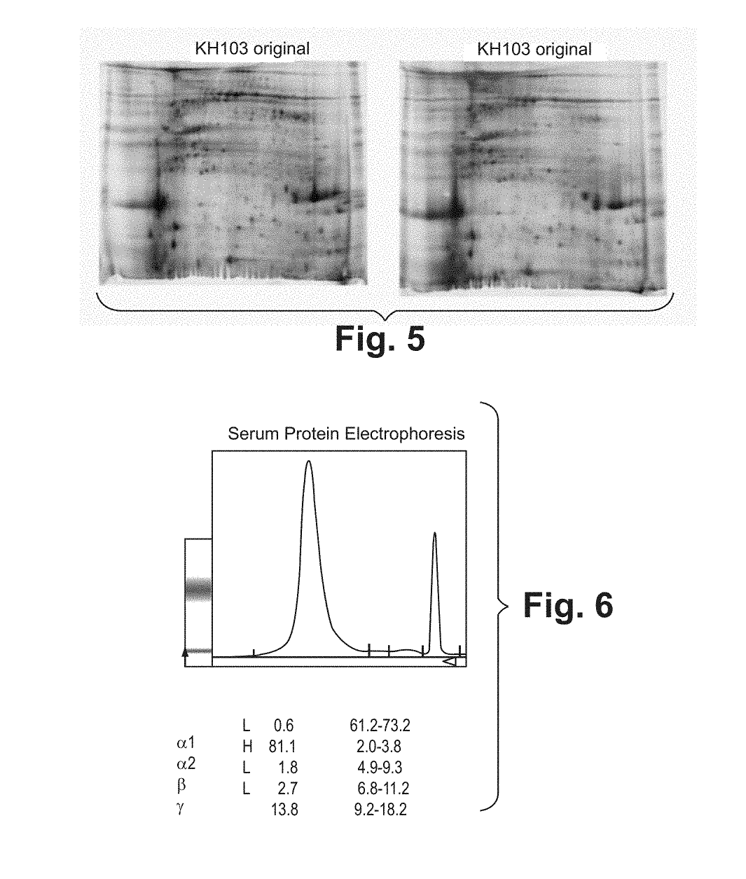

SDS-PAGE analysis was performed in order to detect the molecular weight of the proteins in the mature and immature soy. It was found that soy contains proteins with molecular weights not found in humans. Furthermore, it was found that soy proteins have similar molecular weights to human albumin, human immunoglobulin, and APOA1. It was determined through SDS-PAGE analysis as shown in FIG. 4 that mature soy concentrate and permeate at various concentration levels contains all of the human markers for those proteins.

It was found that mature soy contains 359 distinct proteins. Of the 359 proteins found therein 134 previously undiscovered discovered proteins were sequenced. The proteins are useful for treating certain health deficiencies, diseases, and other health conditions at various concentrations, and large-scale cellular and cell-free production of these individual proteins for such use is encompassed by the present subject matter.

### In Vitro Examination: Serum Protein Electrophoresis

Serum protein electrophoresis of mature soy was undertaken. The analysis as depicted in FIG. 6 shows a high concentration of Alpha 1, Alpha 2, Beta, and Gamma proteins with marked similarity to human immunoglobulin proteins.

### In Vitro Examination: Analysis of HDL, LDL, and TG Levels in Soy

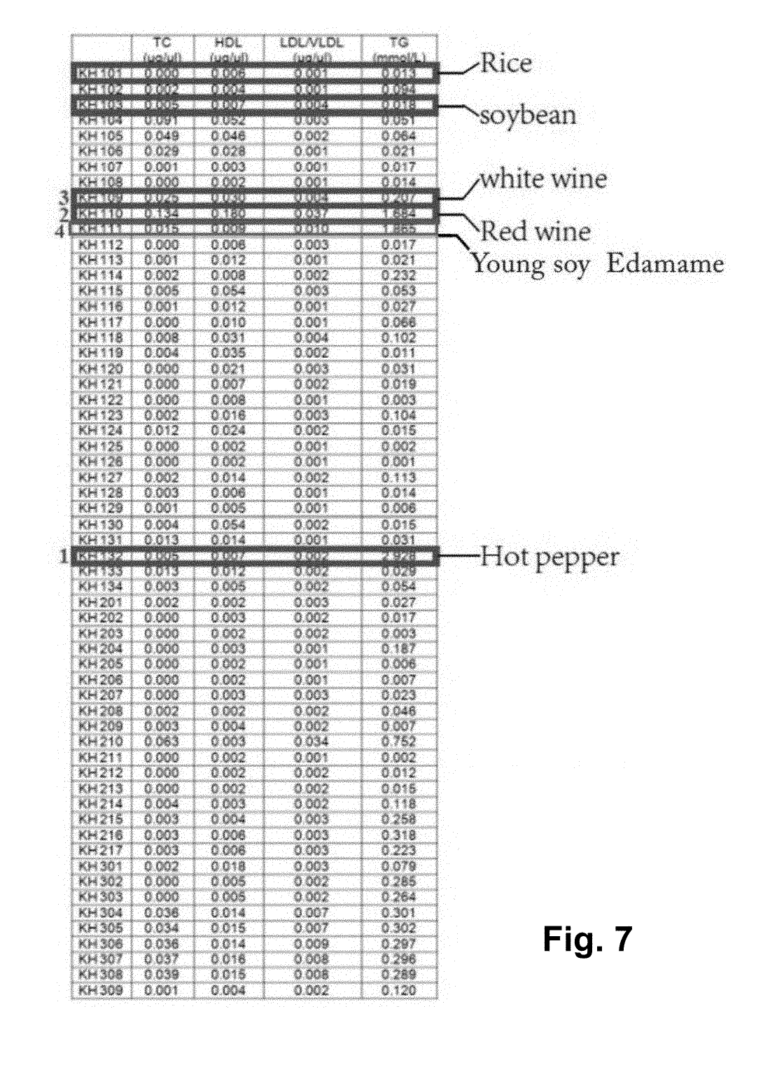

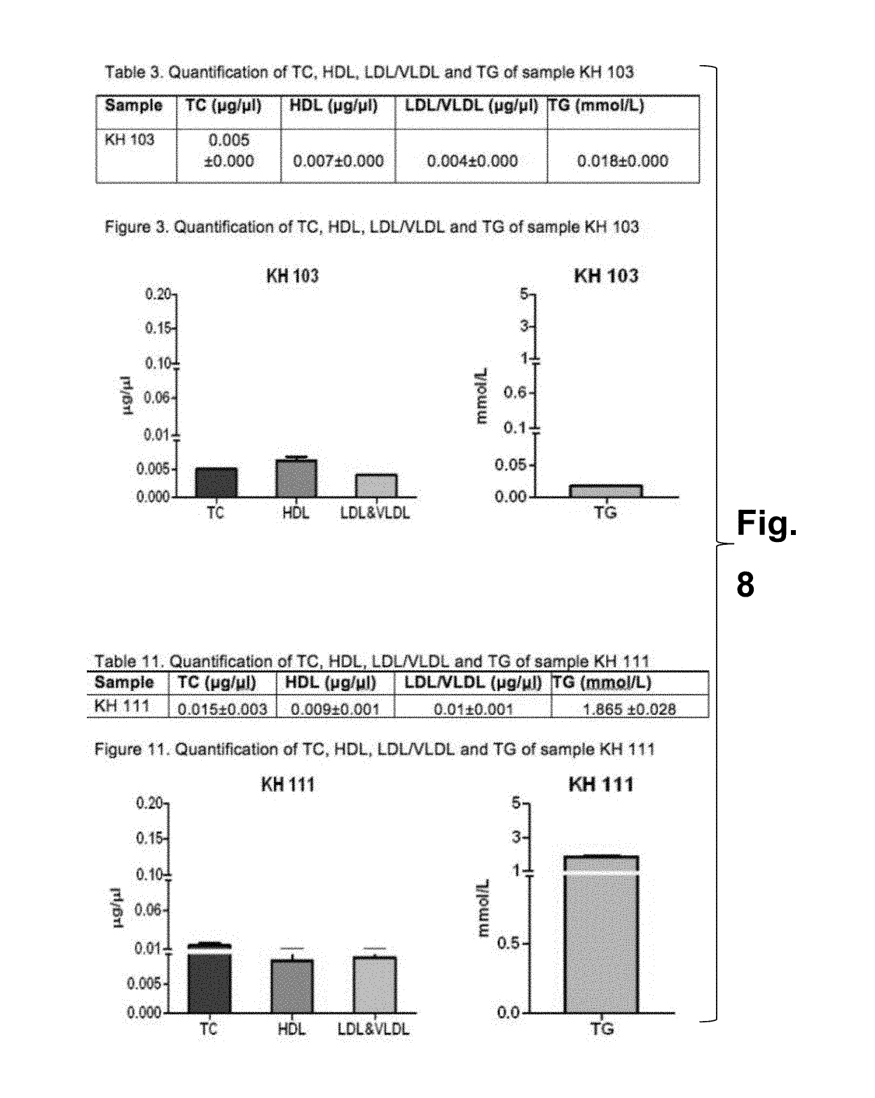

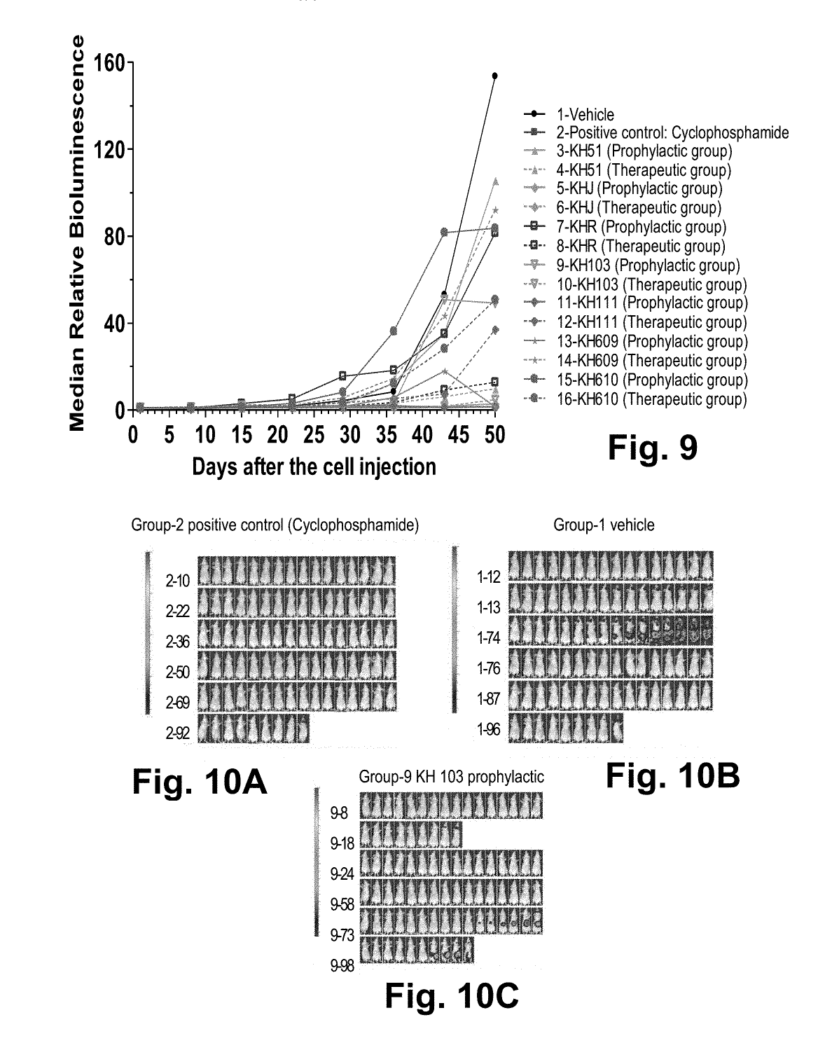

As shown in FIG. 7, immature soy was found to have the second highest level of triglycerides among 100 tested items with 1.865 mmol/L. The highest level of triglycerides of the 100 tested items was found in chili peppers.

### In Vitro Examination: Quantification Levels in Young and Mature Soy

Quantification levels of TC, HDL, LDL/VLDL, and TG were analyzed for immature and mature soy. The results are shown in FIG. 8.

### In Vivo Examination: Inhibition of Leukemia Proliferation

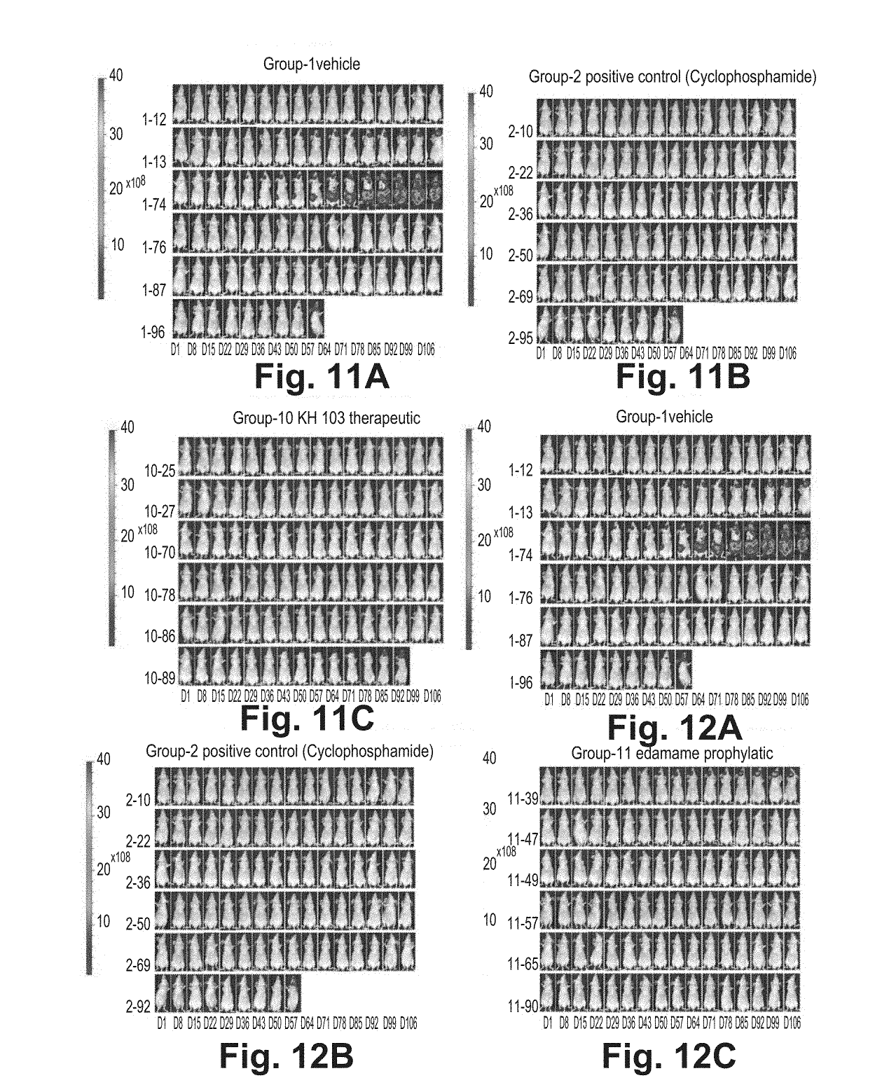

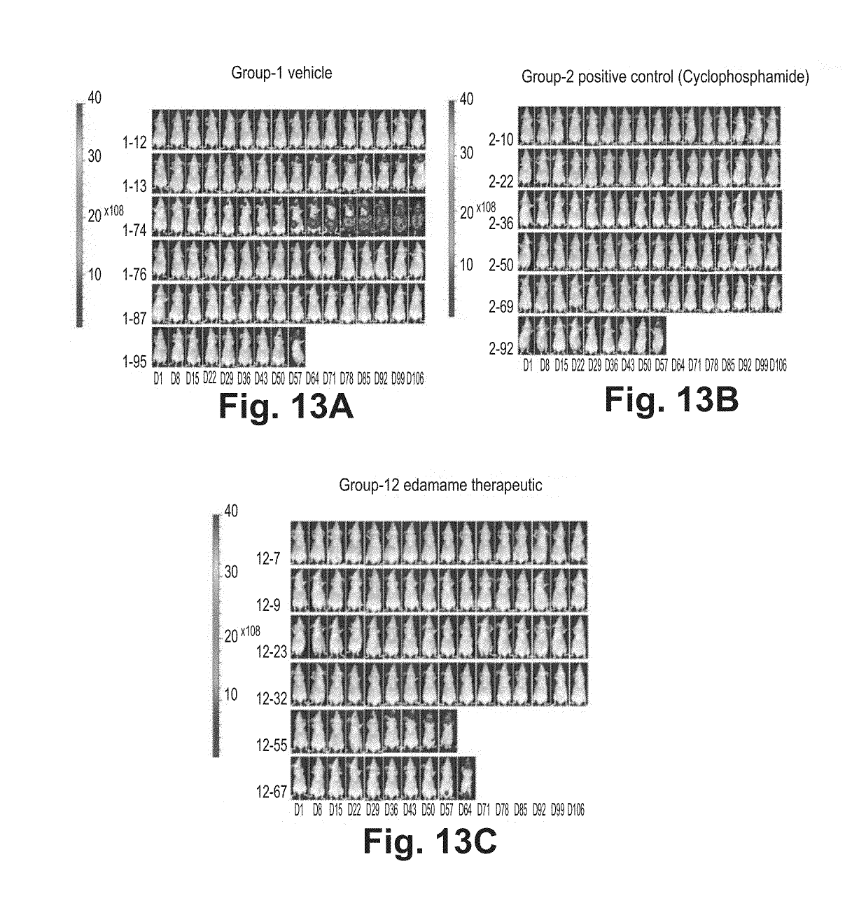

Leukemia inhibition animal trials of immature and mature soy constructs containing one or more of the previously undiscovered and discovered 359 soy proteins were conducted. The results of those trials are shown in FIGS. 10-13. It was discovered that animals in the therapeutic soy-treated groups had decreased proliferation of leukemia cancer cells compared with the cancer proliferation of animals in the vehicle control groups.

### Testimonials

An embodiment of the present subject matter is directed to a combination of at least 350 proteins, among which 135 proteins are newfound proteins.

In an embodiment, the present subject matter is directed to unnamed protein products, uncharacterized proteins, and Putative uncharacterized proteins found in different products. Products KUNAKIN™ and KUNKAKINMIN™ are made of soy component in the present subject matter. Upon recent research conducted by Wuxi AppTec, it was found the KUNAKIN™ was effective in inhibition of H1N1, H3N2 and HCV virus or its replicon.

In an embodiment, the proteins are synthesized by KH good healthy cells, which can send signals to the damages, sick, and bad cells to triggers synthesis of good proteins to transform these cells to become good healthy cells; send signals to other currently undamaged cells to synthesize good proteins to protect them from being damaged, infected and prone to DNA and other cellular alterations; and send signals to the body to produce new cells that are healthy and to forbid them from being affected by intracellular and extracellular damaging signals.

An embodiment of the present subject matter is directed to soy proteins separated by a proprietary method into many fractions, wherein the proteins have a molecular weight similar to proteins in humans. Albumin immunoglobulin and high density Lipoprotein, or APOA-1, for manufacturing from plasma and the mechanism is described. The same molecular weight proteins of albumin immunoglobulin and APOA-1, and all other 135 newfound proteins in soy as proven through SDS Page test were found in soy has more proteins than from human plasma.

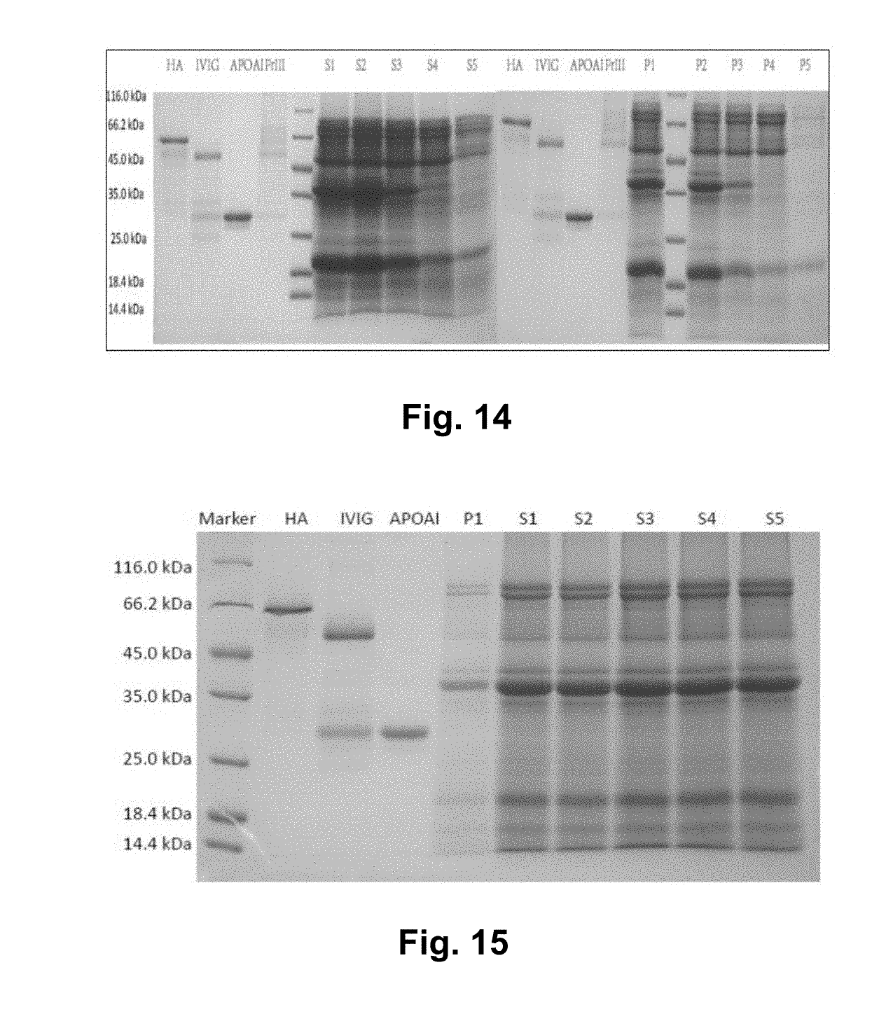

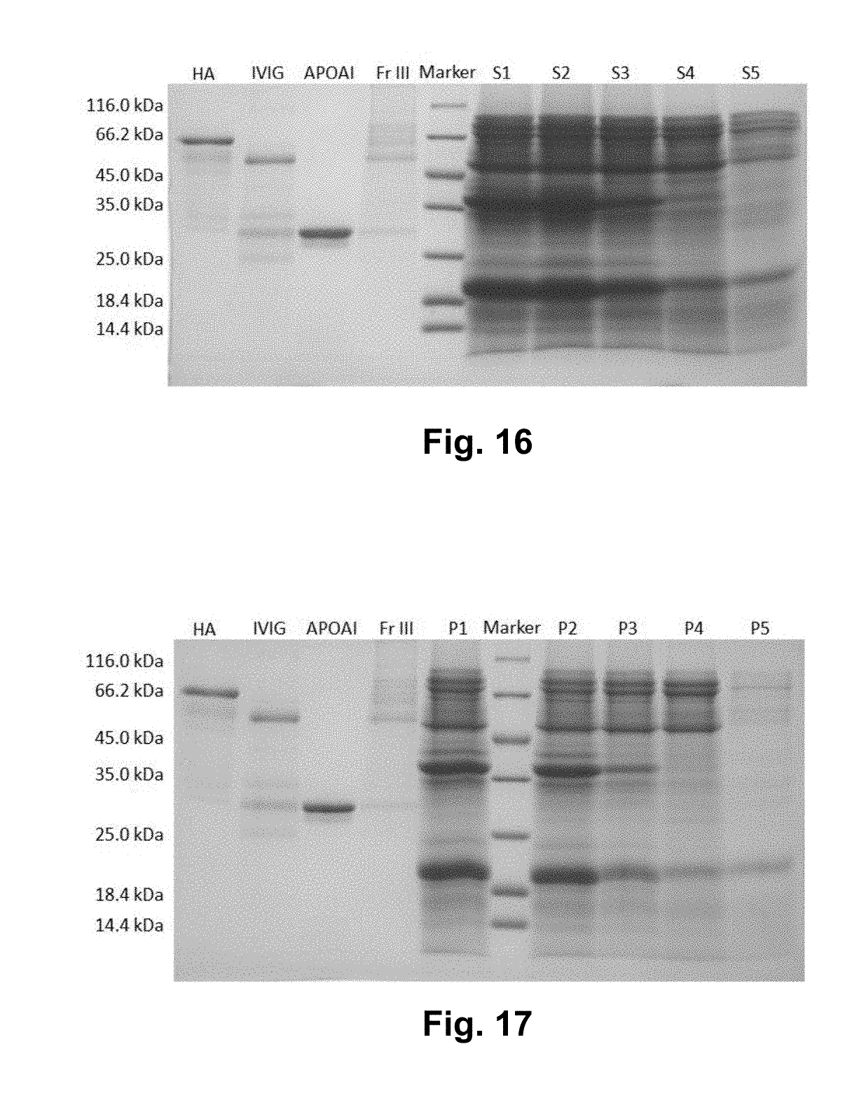

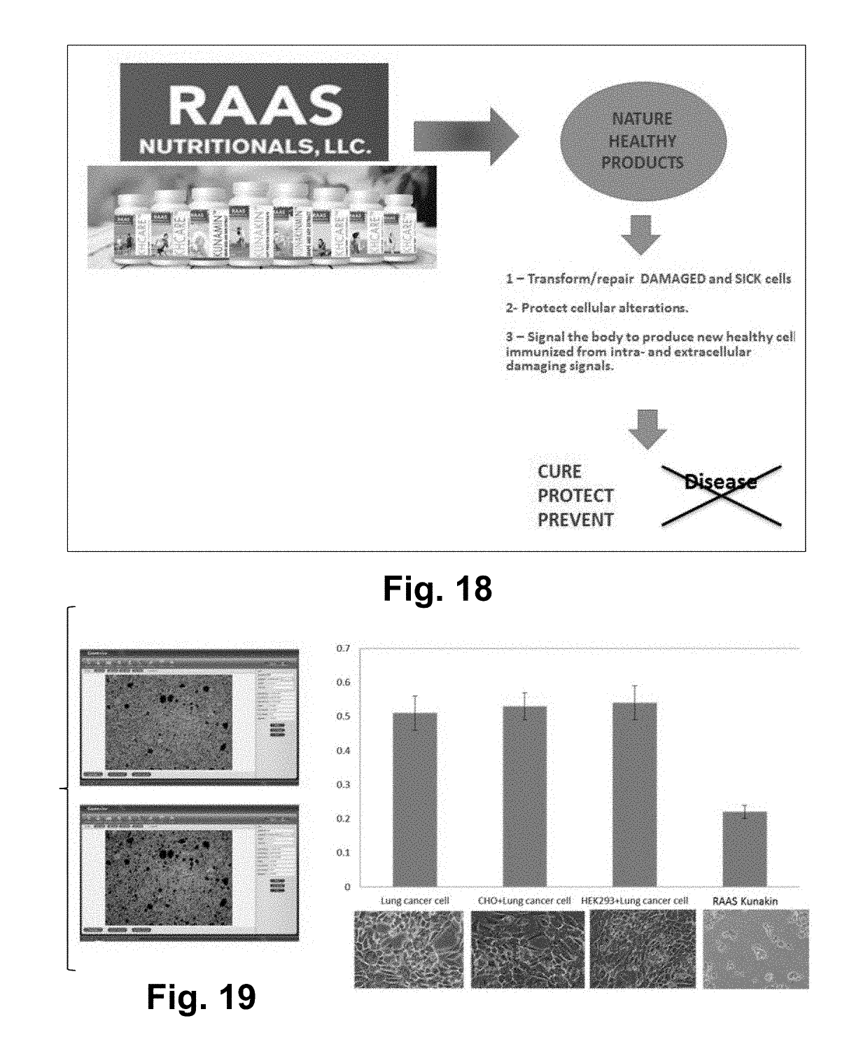

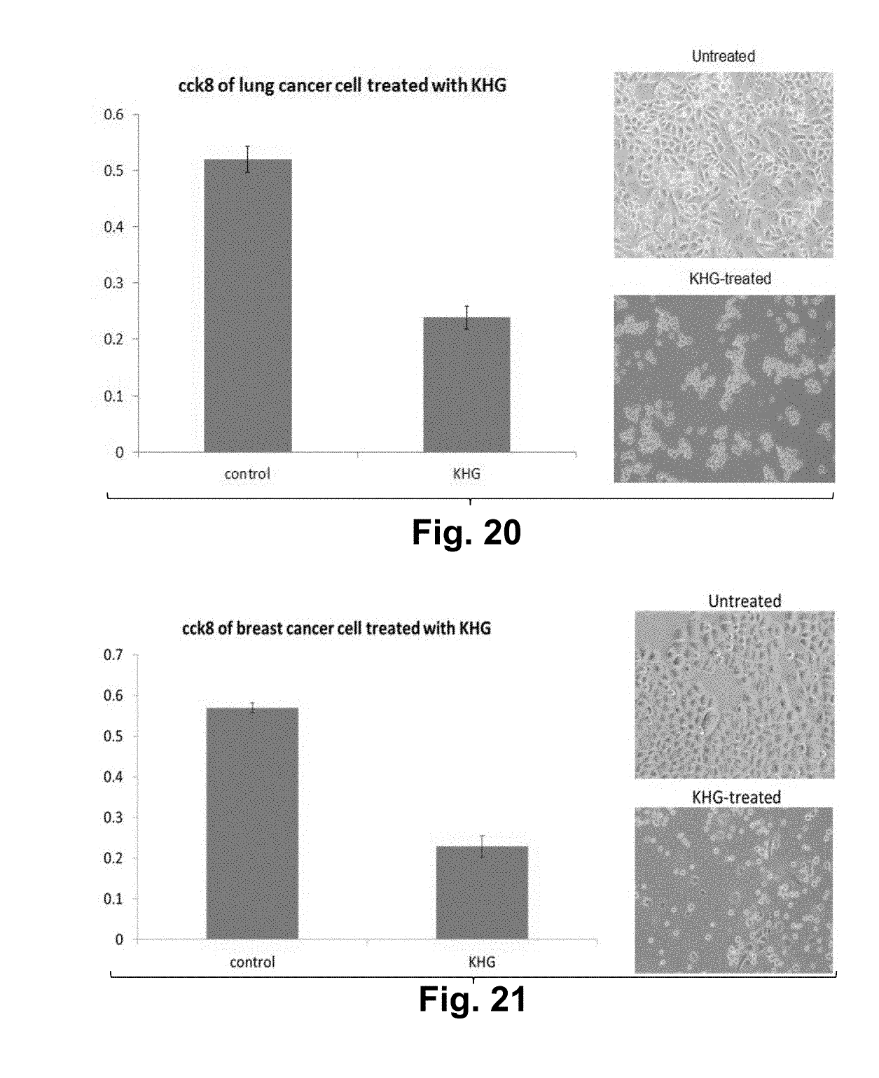

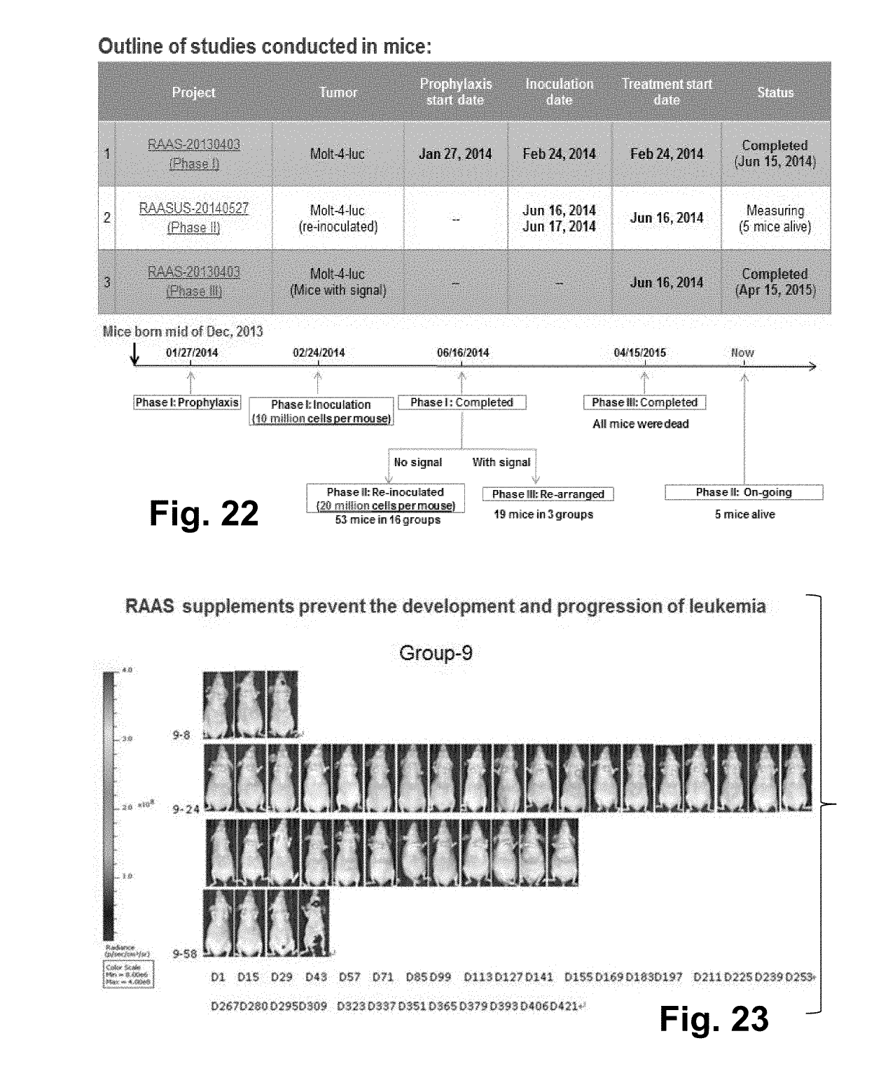

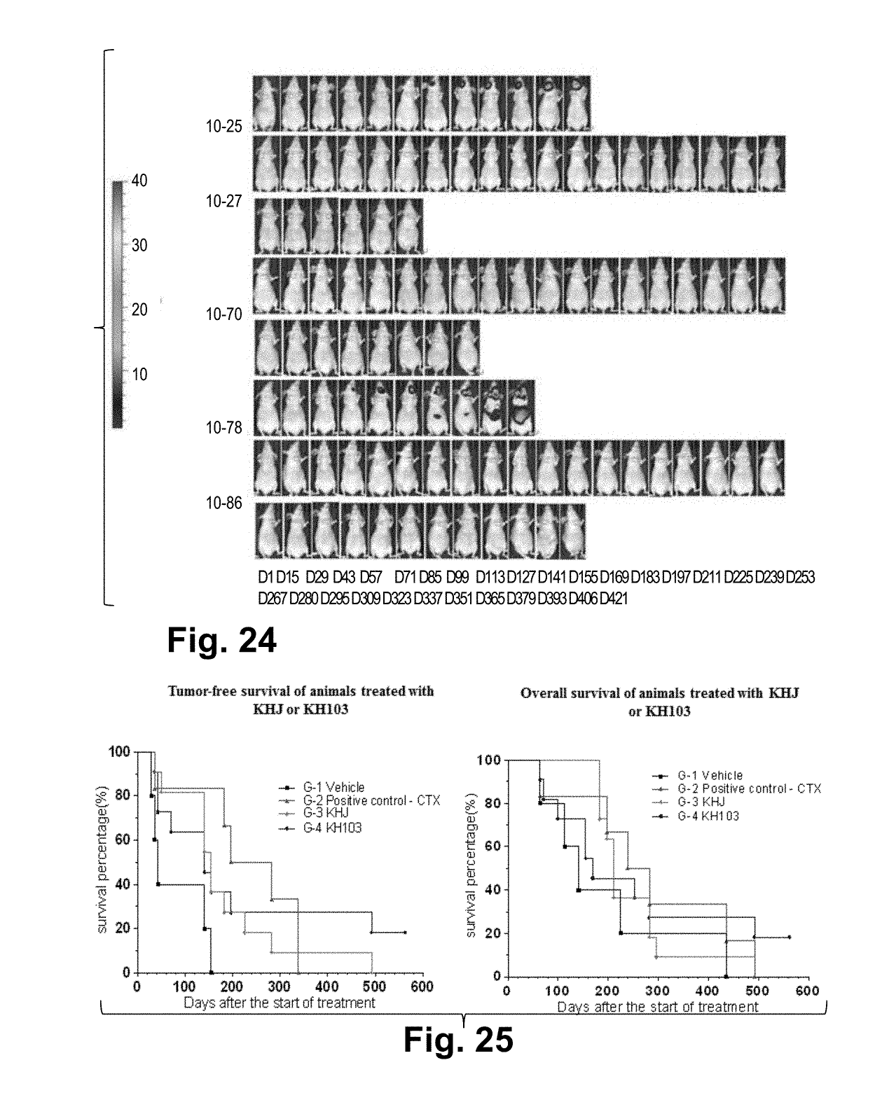

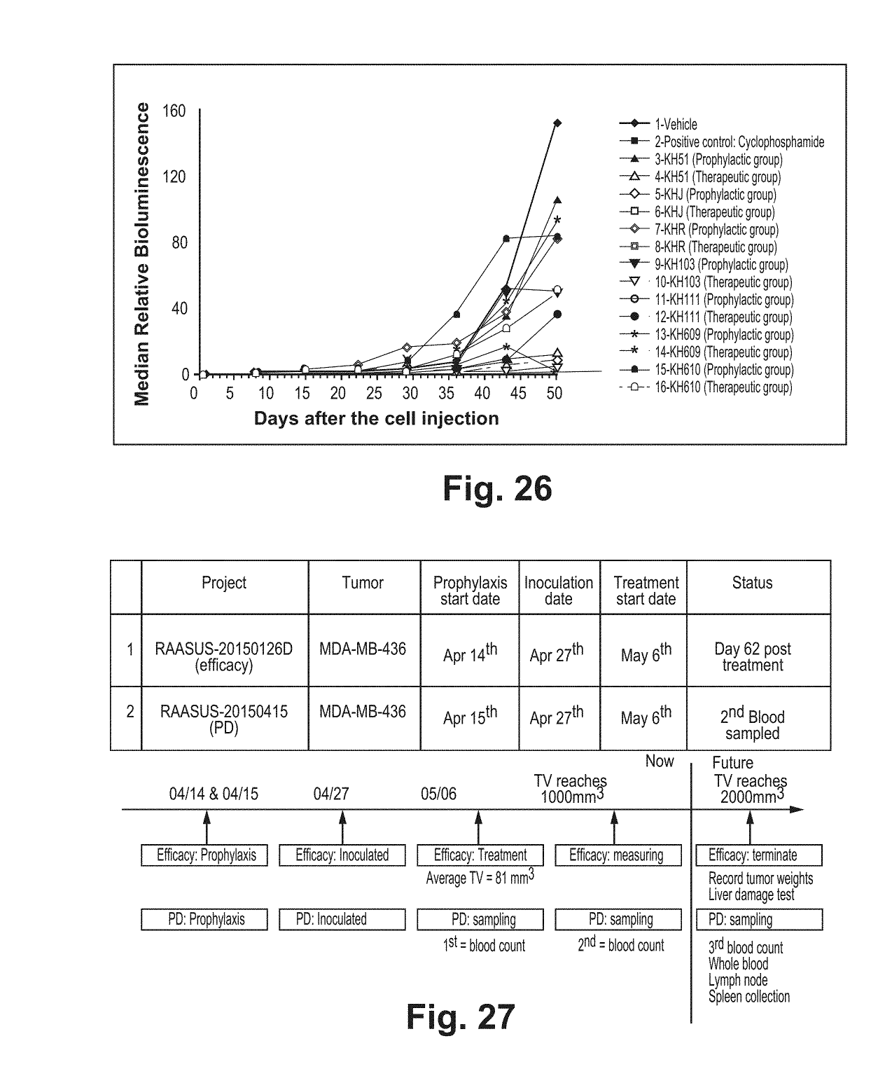

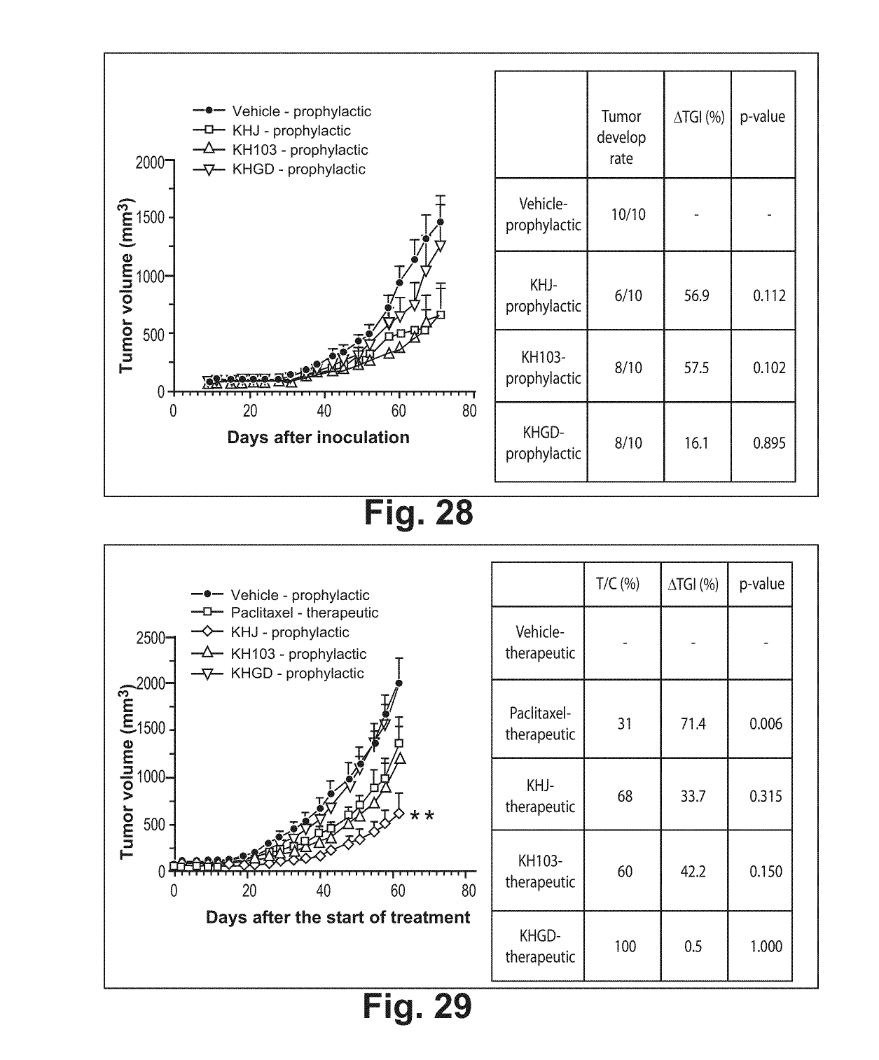

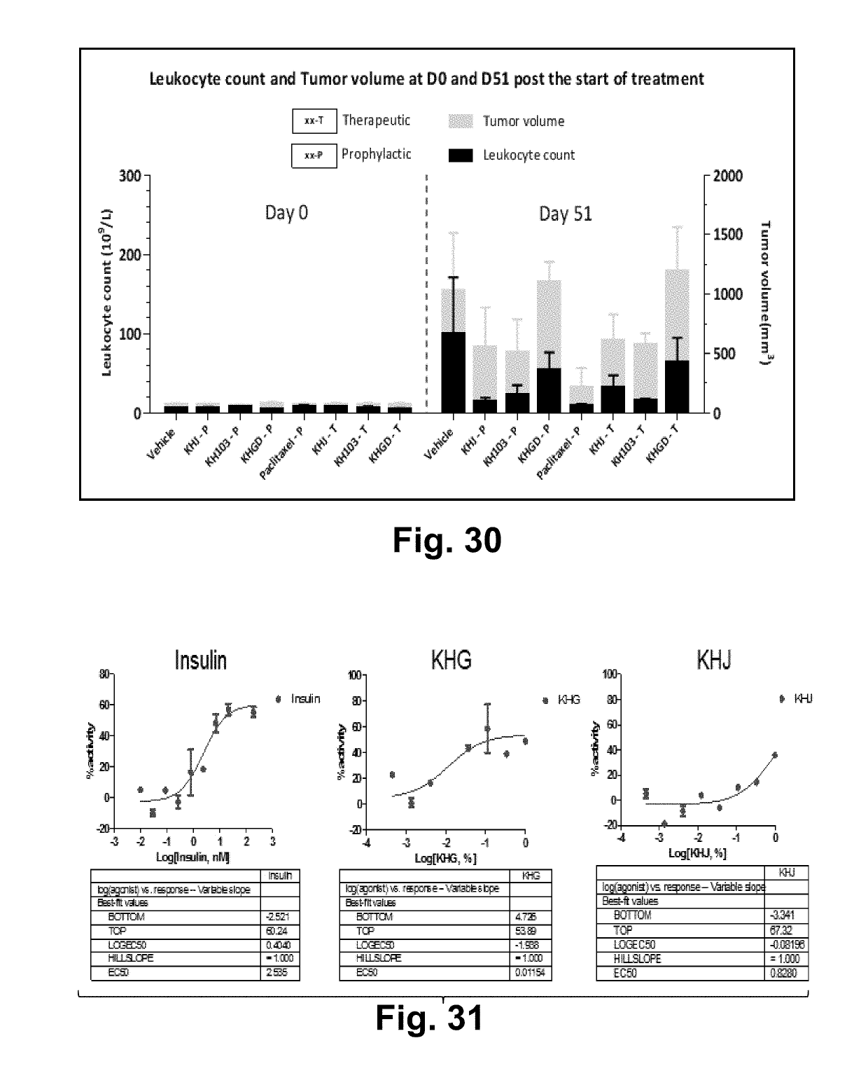

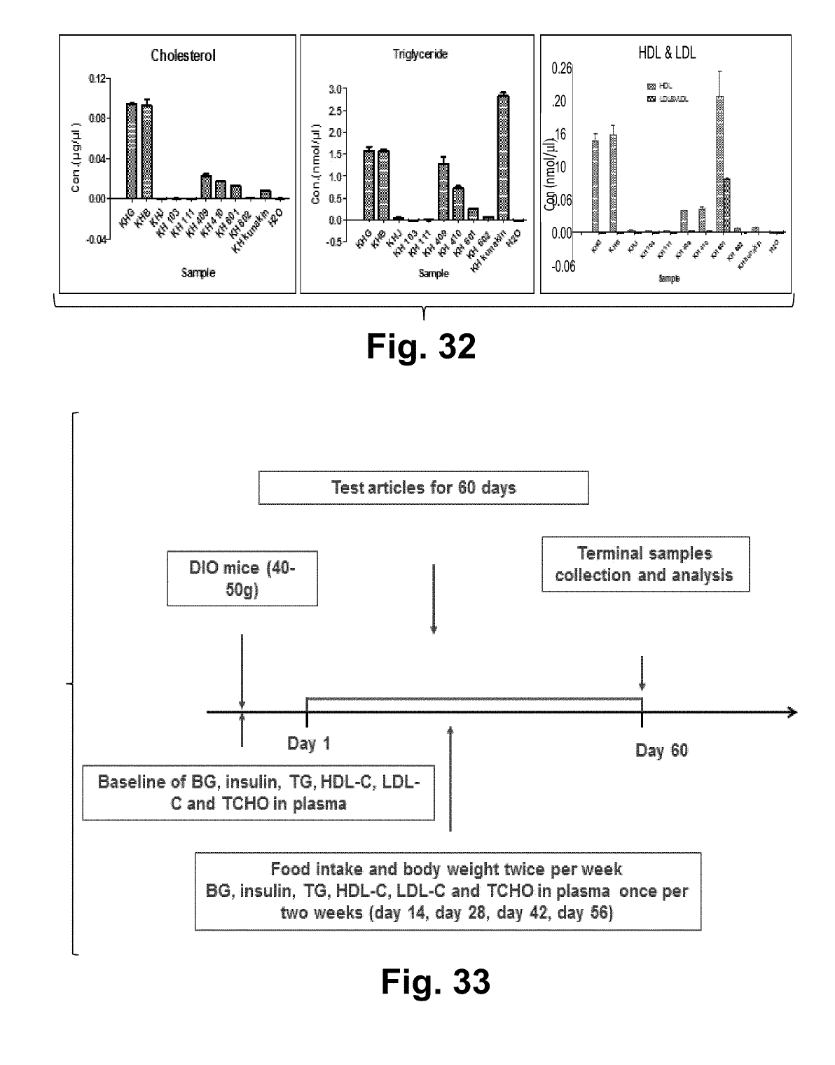

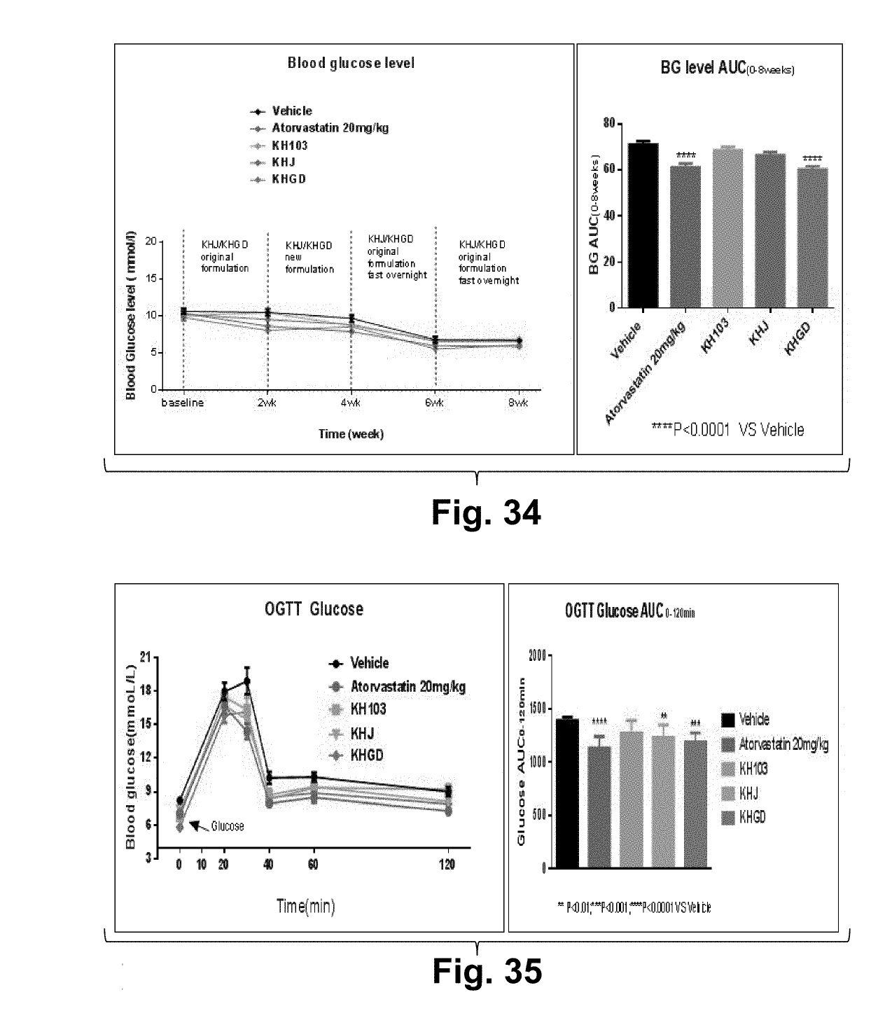

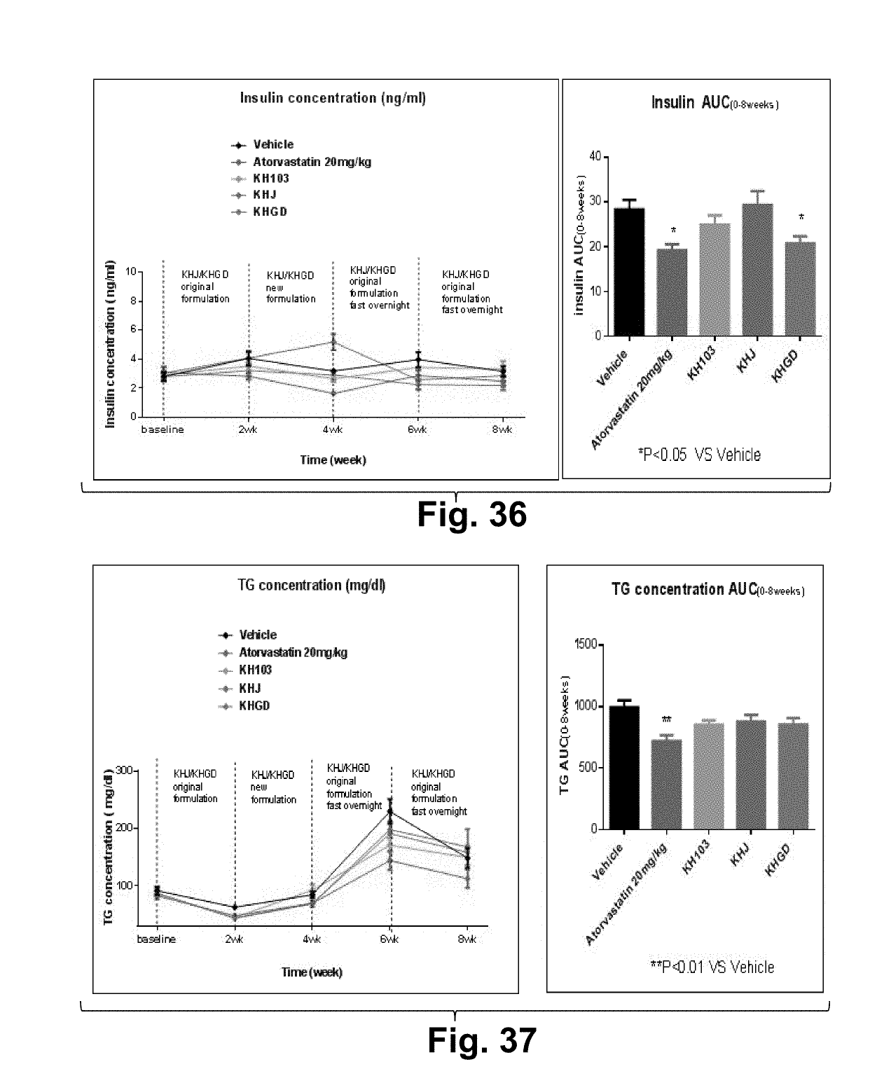

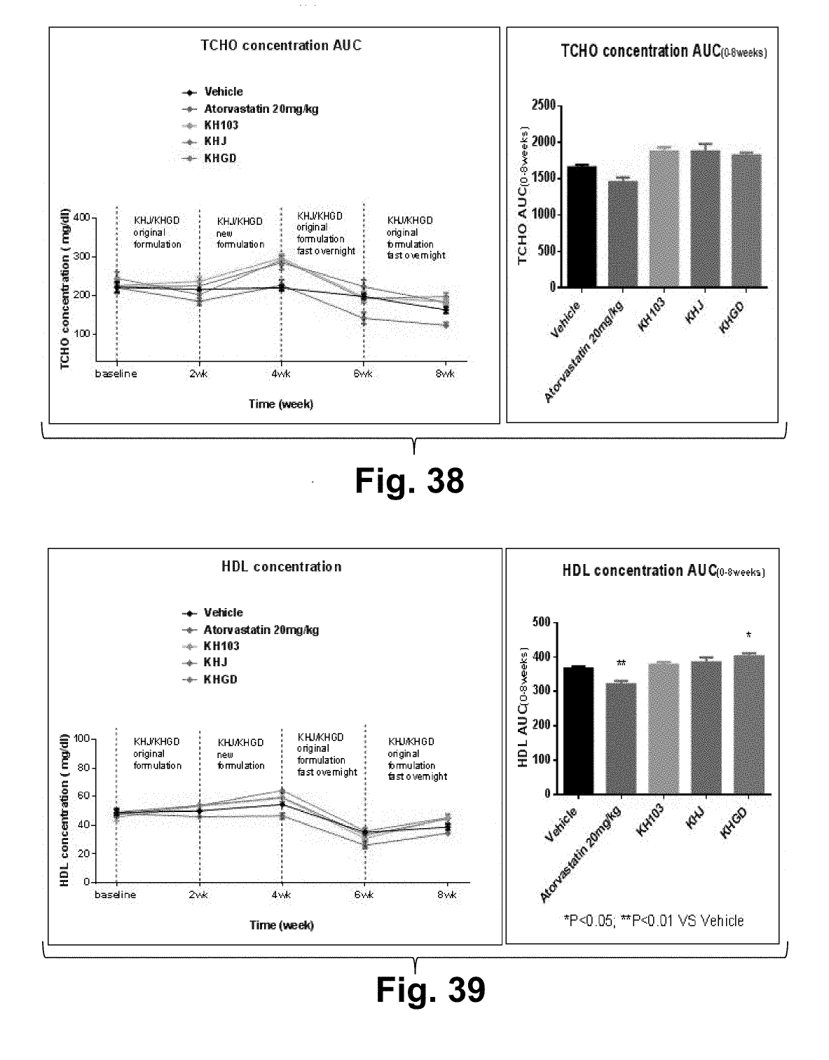

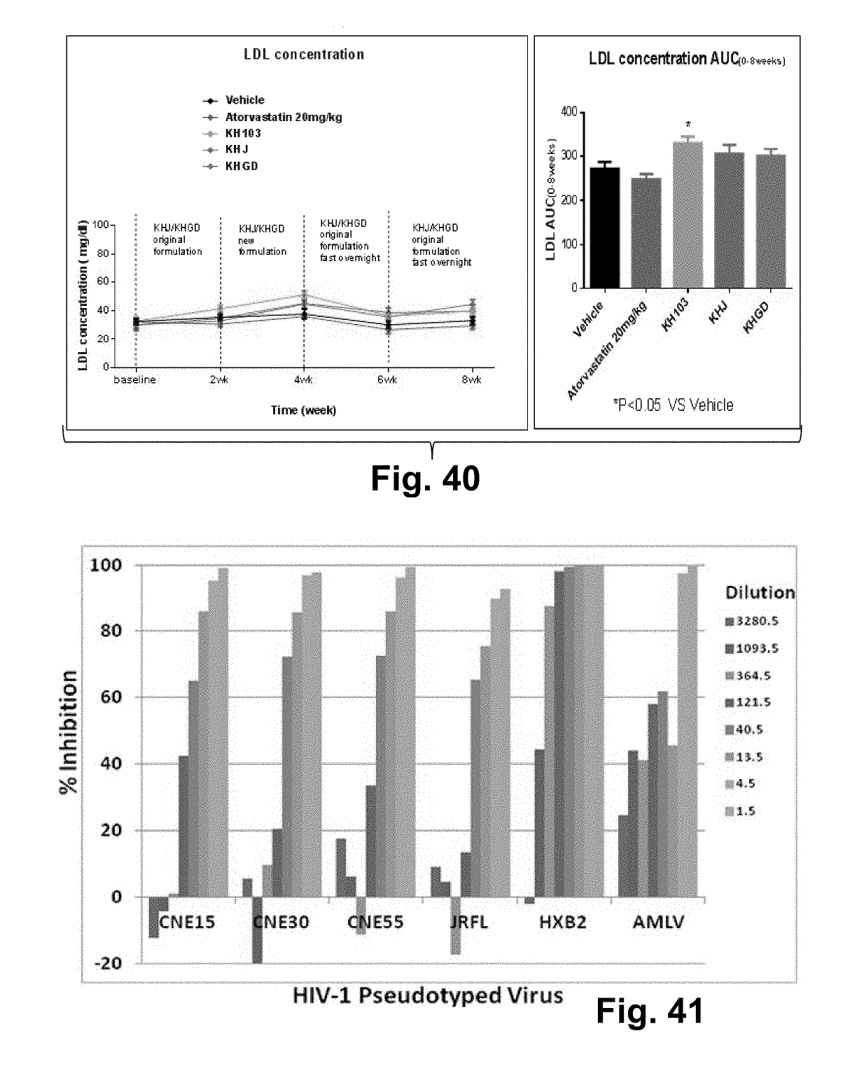

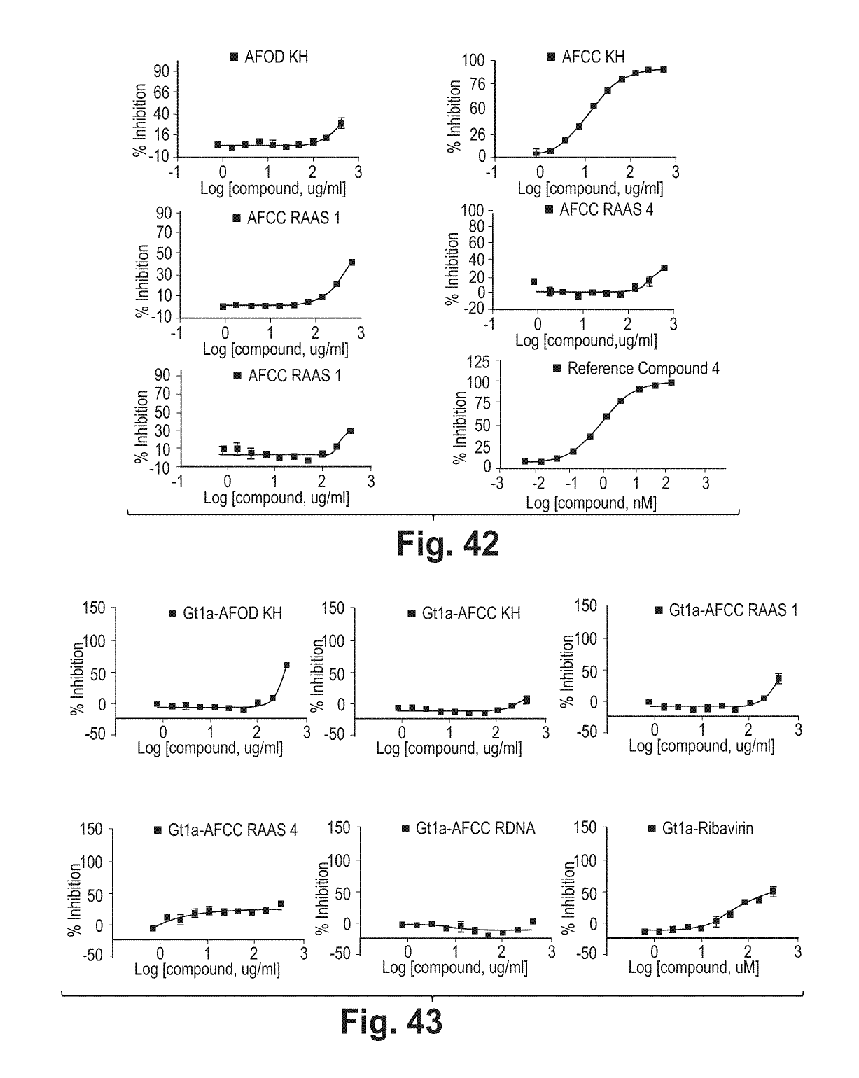

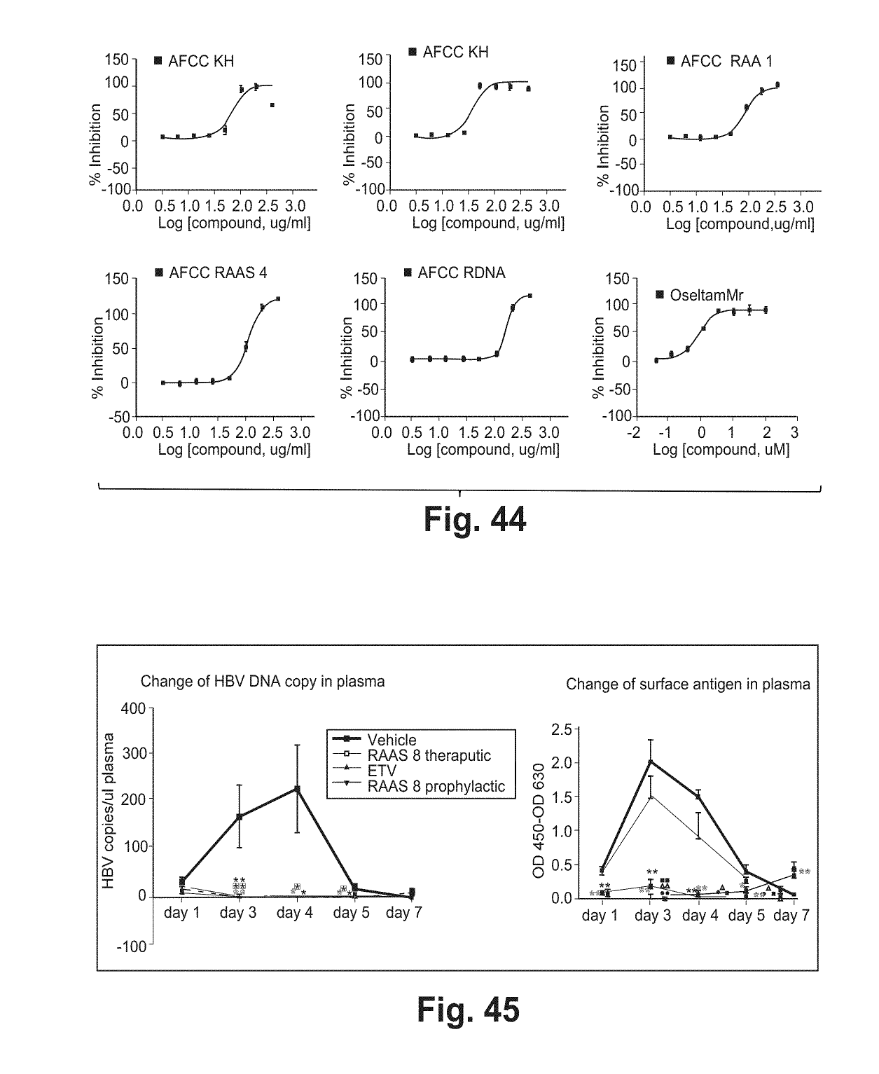

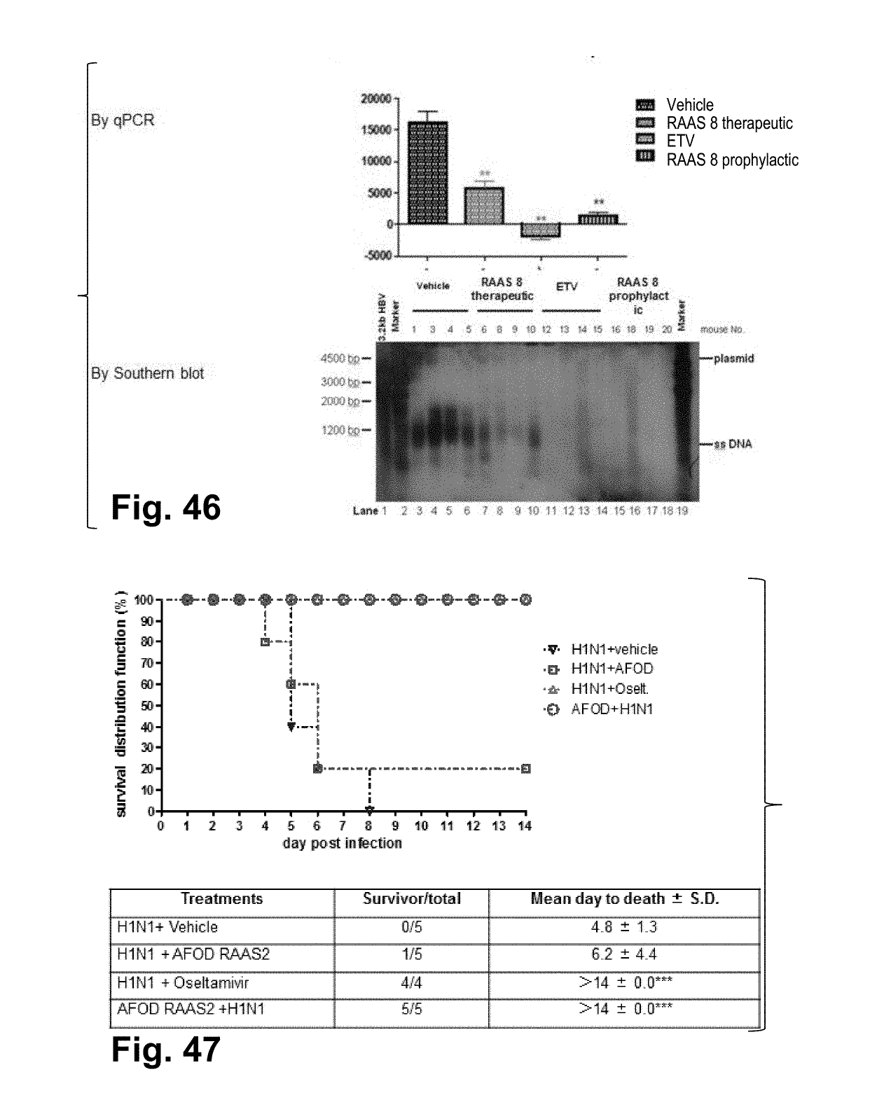

FIGS. 14-17 show SDS Page results for soy. From cryopaste fractions 1, 2, 3, 4, and 5, approximately 155 proteins were found, among which 55 are newfound proteins and cells in humans with 100 existing proteins. In soy, 355 proteins were found and combined with 135. Products may be from plasma, as one protein is not powerful enough to cure diseases and cancers, as indicated by Baxter in which intravenous immunoglobulin was used to “cure” Alzheimer's, but failed because it was only one protein.

In the soy proteins, the processing comprises separating the fractions obtaining the best and then combining at the end at least 350 proteins into one product. The product is trademark KUNAKIN™ with powerful potency shown in in vitro, in vivo, and in human observation for cardiovascular diseases, chronic kidney diseases for which there is no medicine for treatment, diabetics, inflammation, stone, chronic lymphoma leukemia, breast cancers, lung cancer, liver cancer, Alzheimer's, Hepatitis C, Hepatitis B, and spinal injury, wherein the aforementioned cases are only for medical observation.

The products from soy not only cure, but also prevent, cancers and diseases. Furthermore, the products may be used as prevention instead of vaccine in the case of an outbreak of viruses such as the Bird Flu h1n1 that happened in Mexico in 2011, which costs economic losses, h2n1, h7n9, ebola, SARS in China (Hong Kong in 2003), and MERS in Korea.

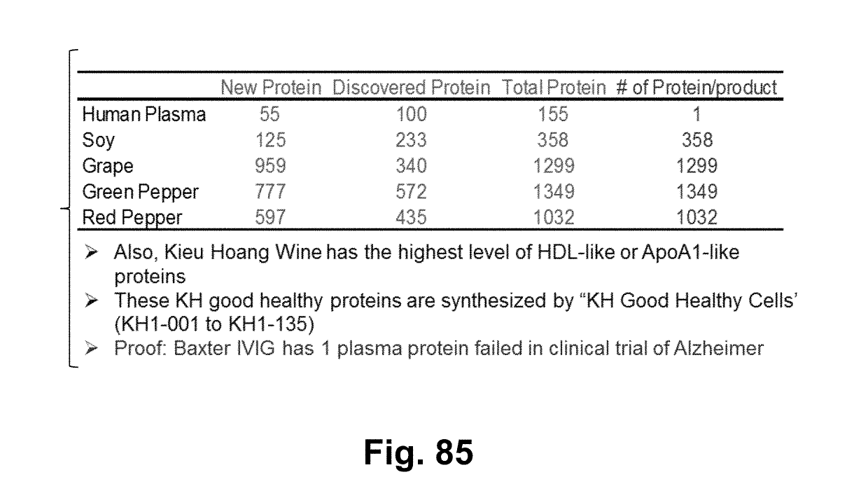

The in vivo studies for leukemia, breast cancers, and diseases such as Hepatitis B show positive results of working in animals. As such, friends and relatives continue to use products to prevent in the long term, and a few cases have lasted for years. Limited trials in friends and relatives both in the USA and in Vietnam have shown positive results. FIGS. 18-85 show testimonials.

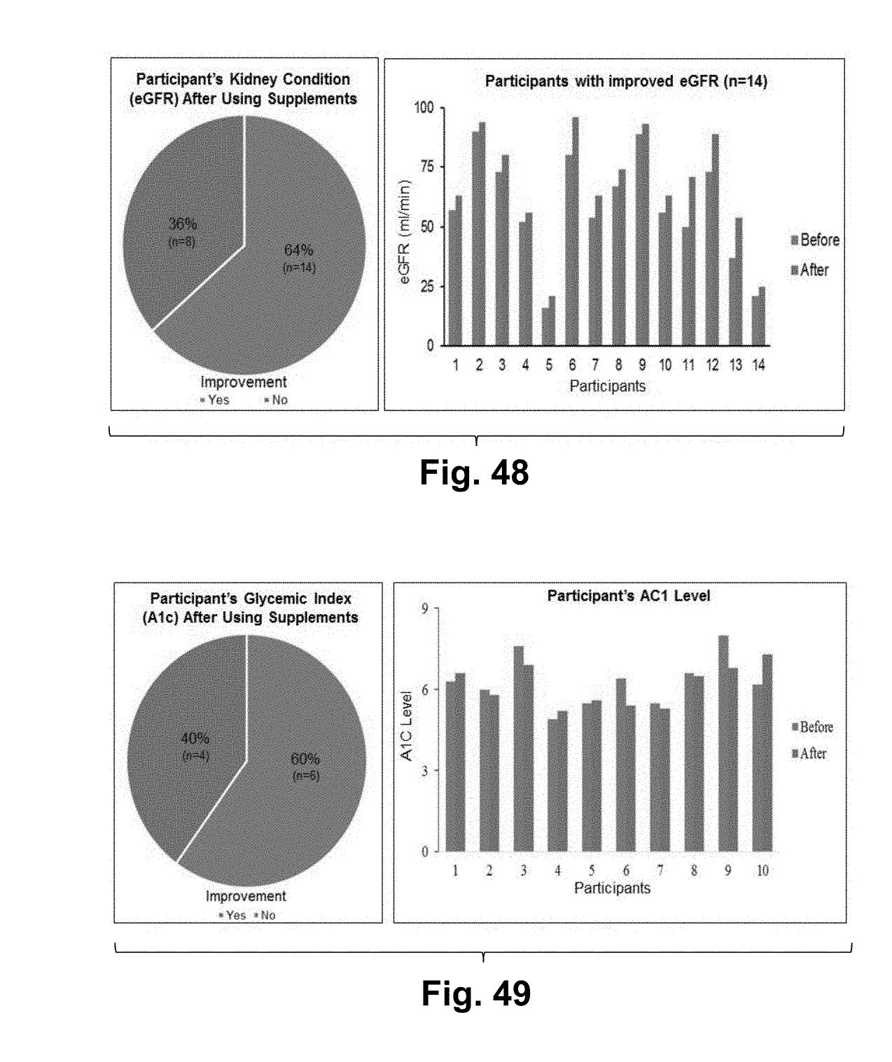

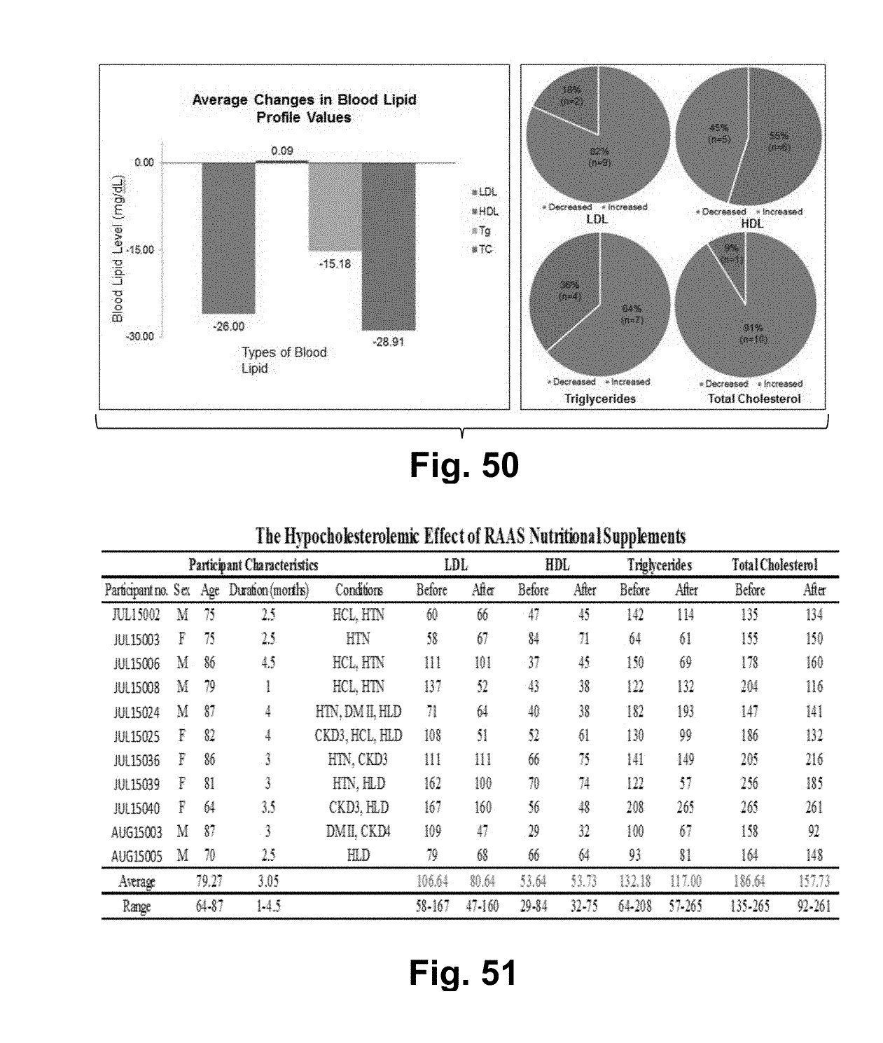

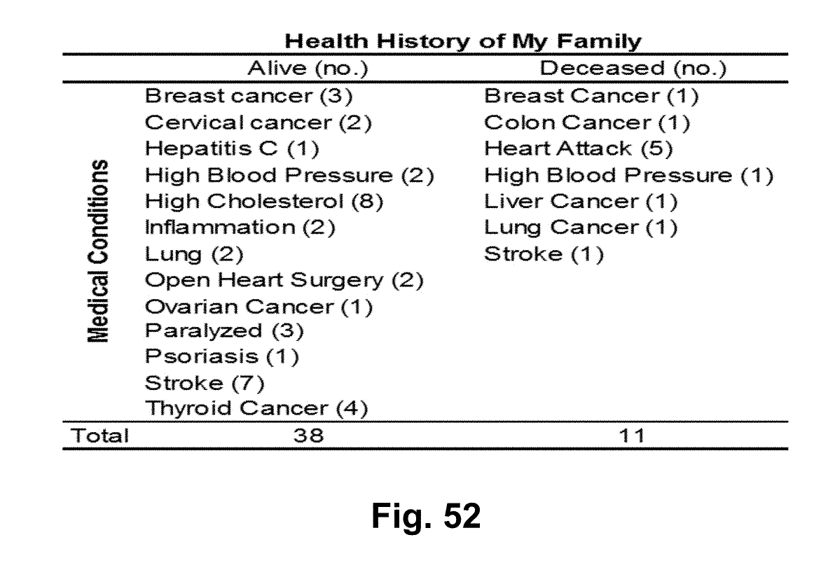

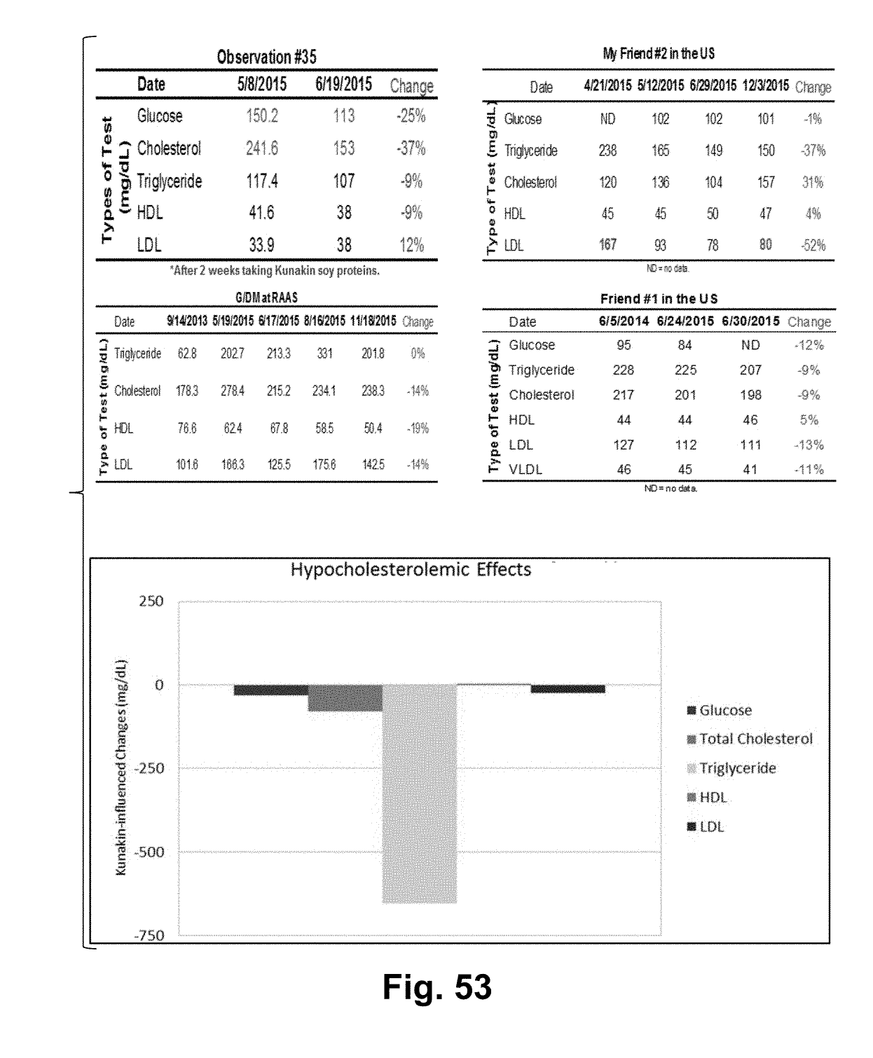

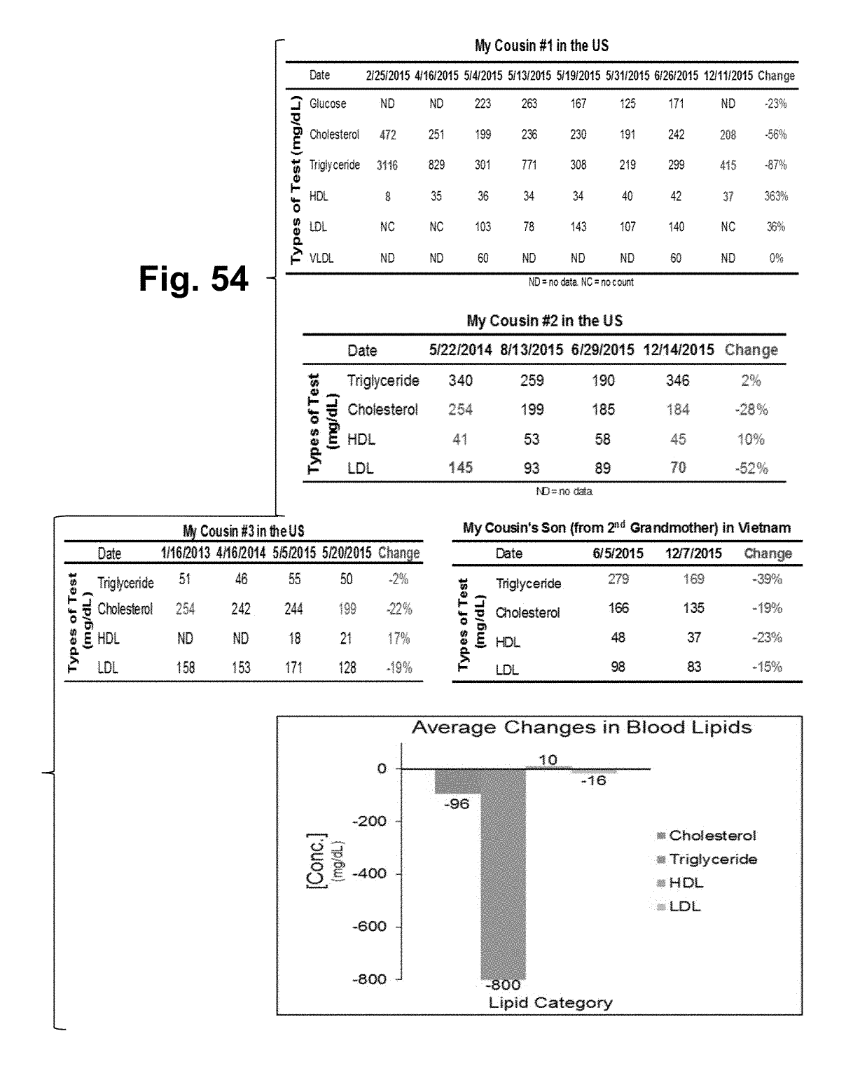

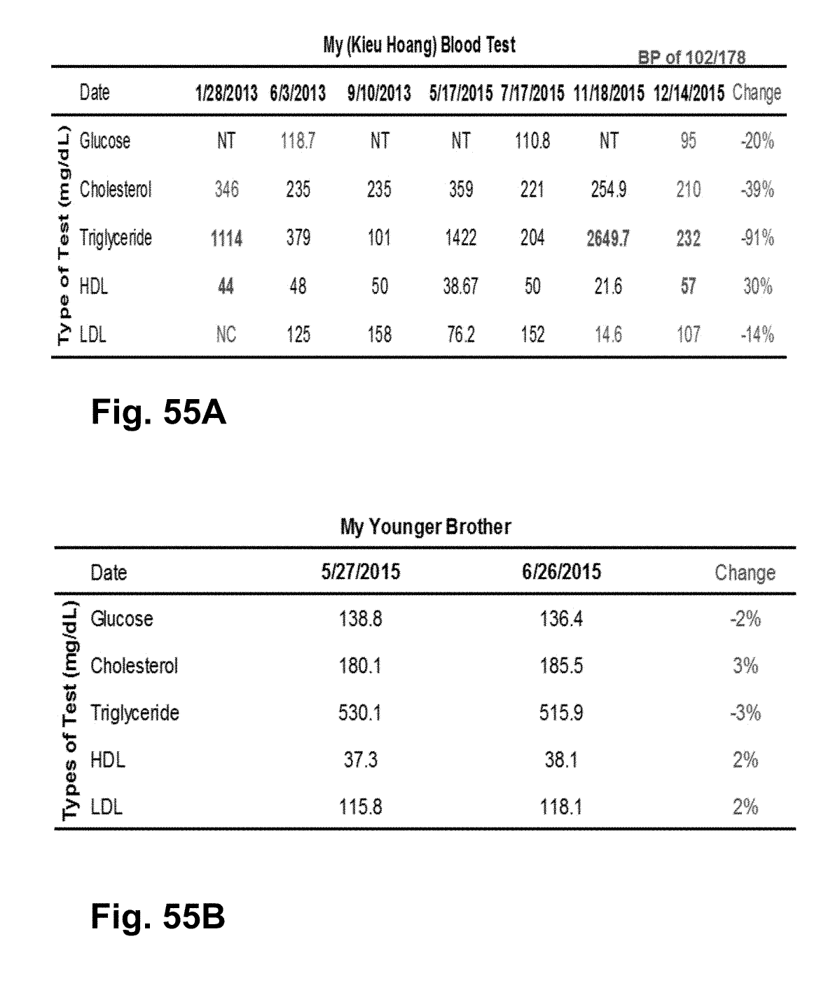

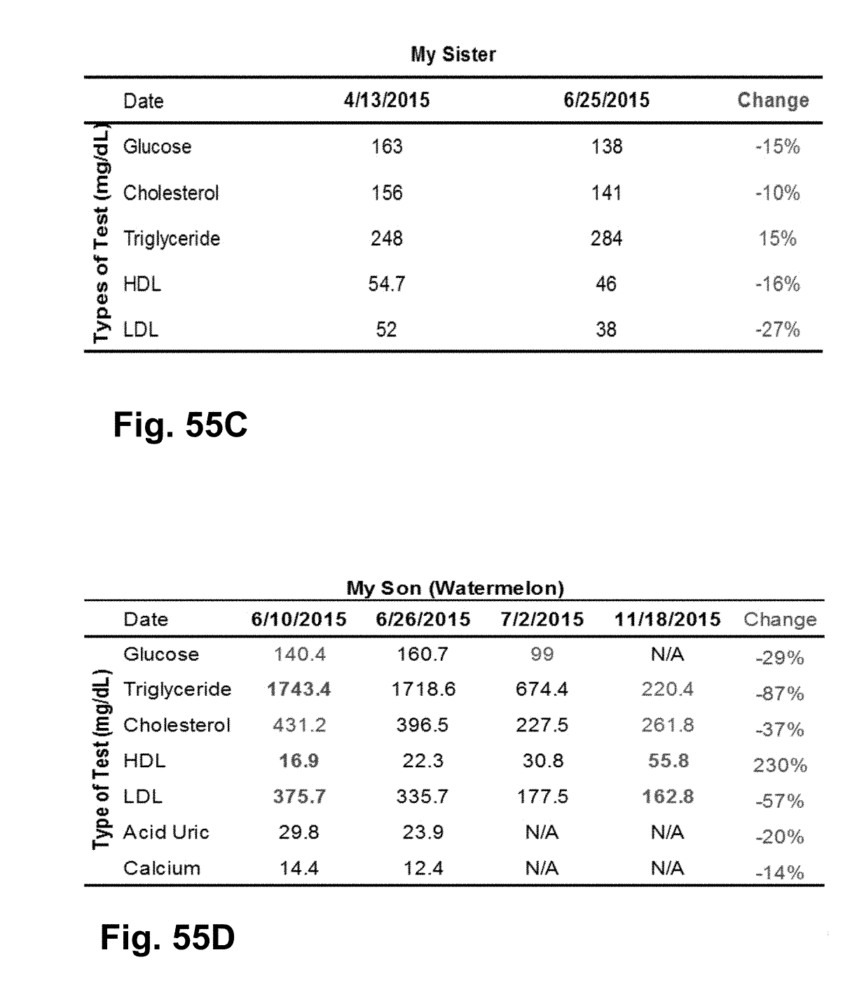

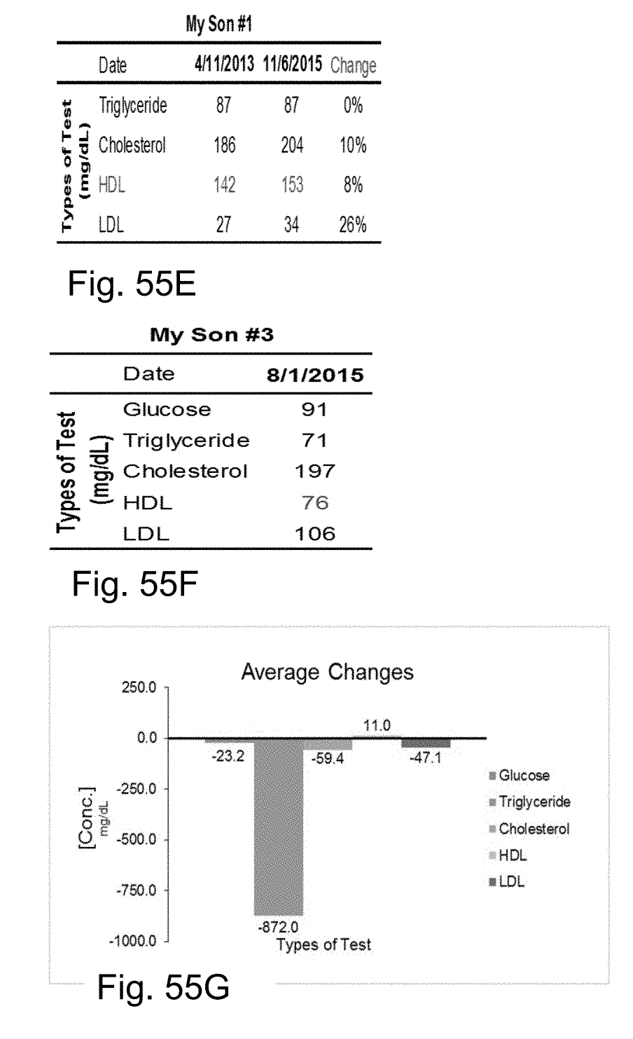

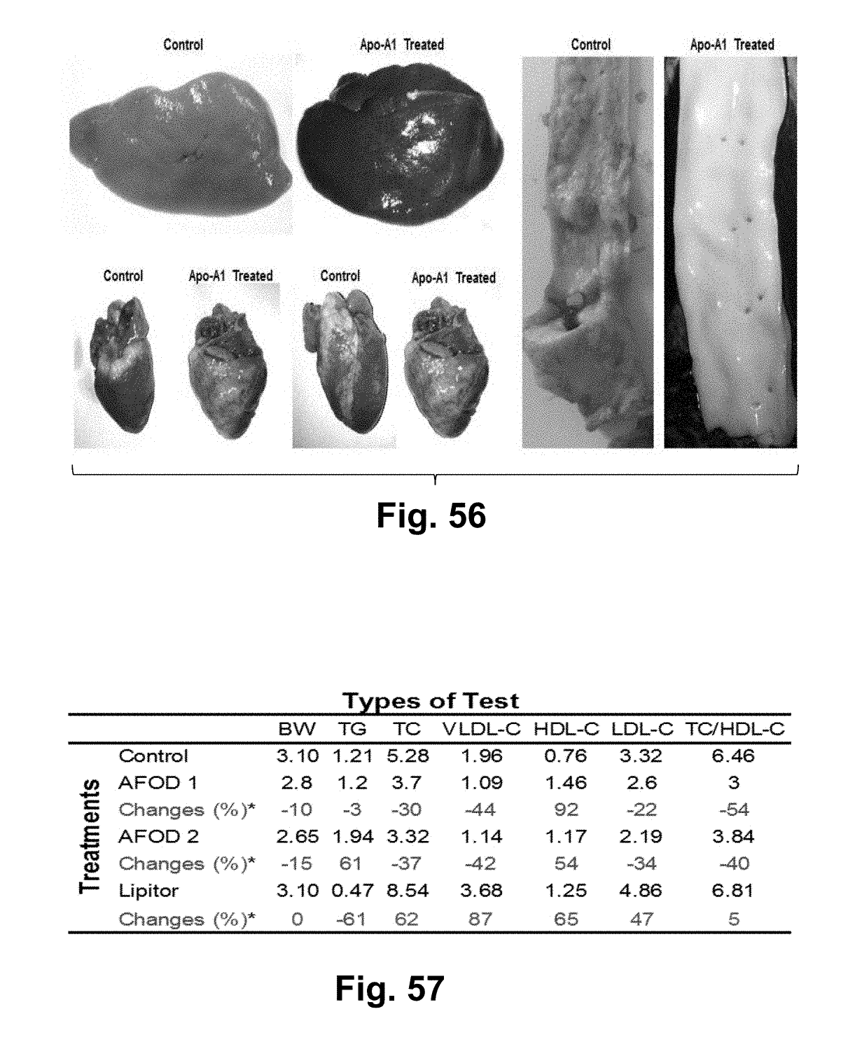

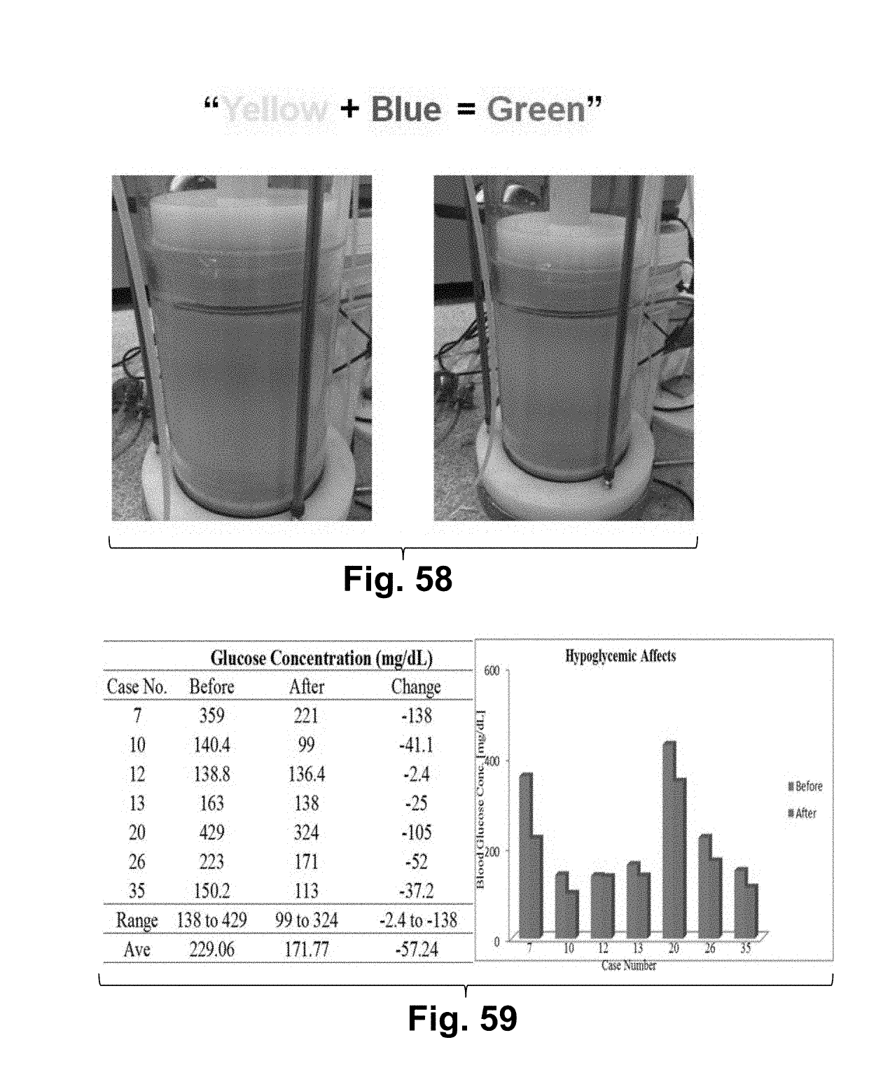

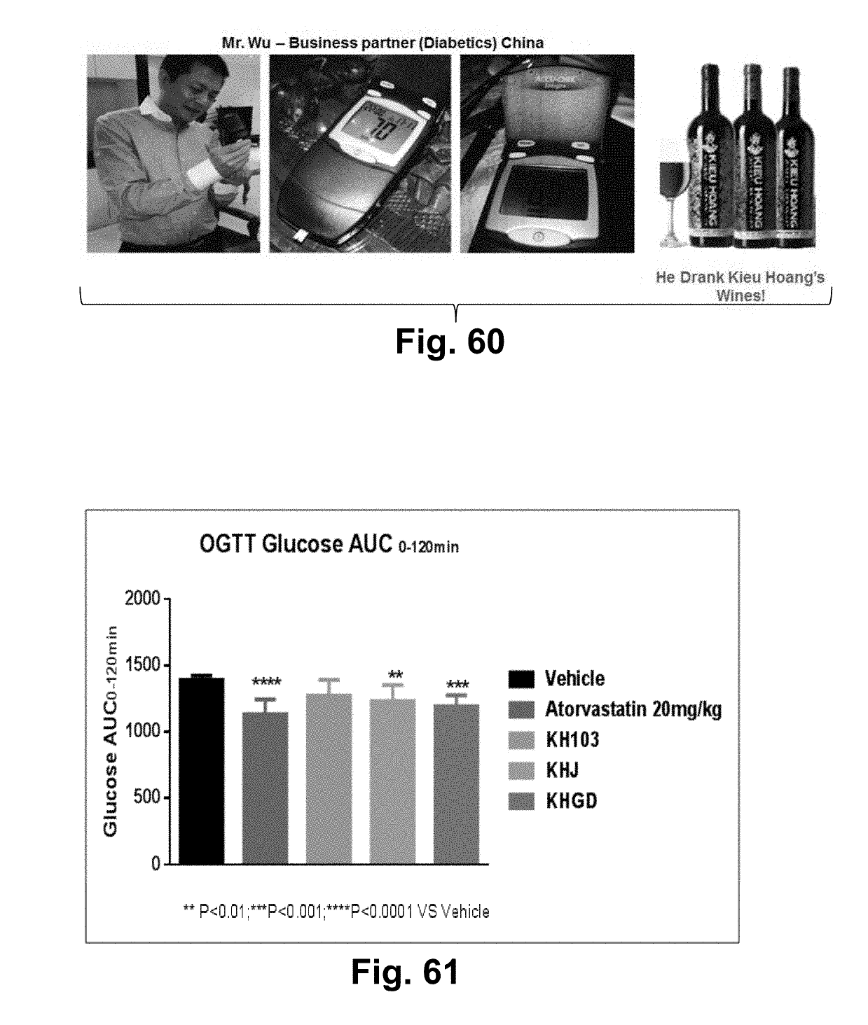

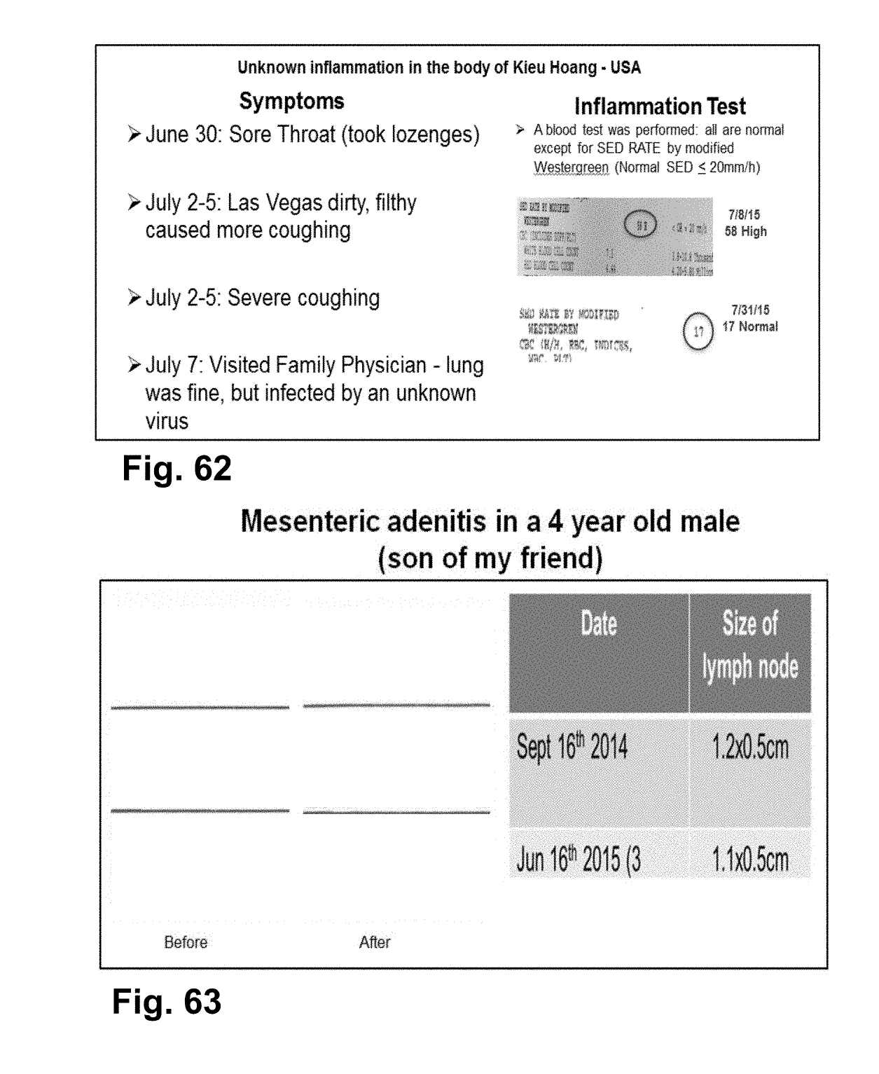

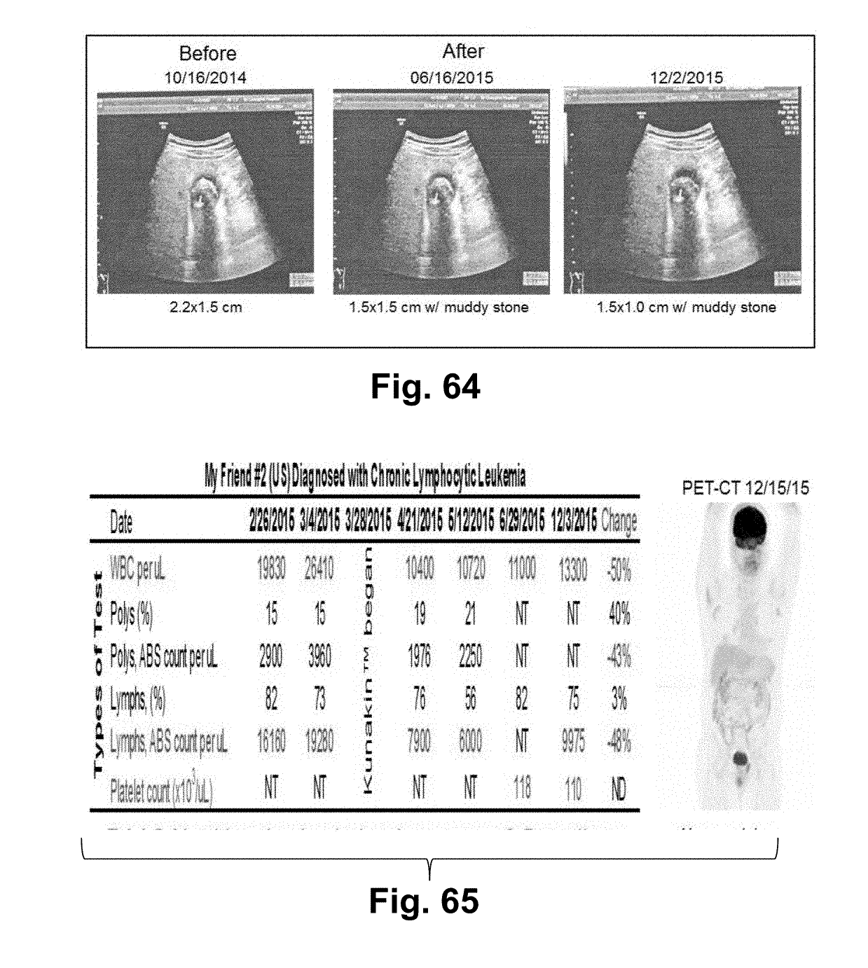

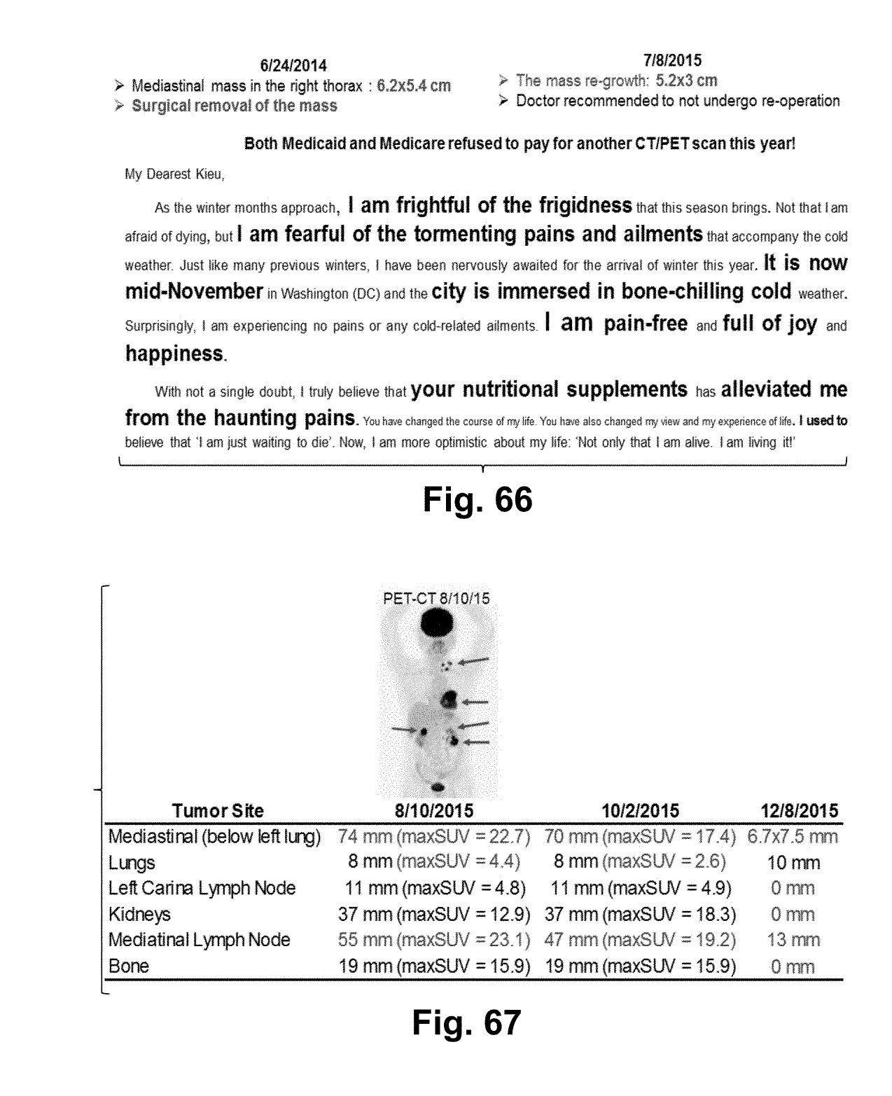

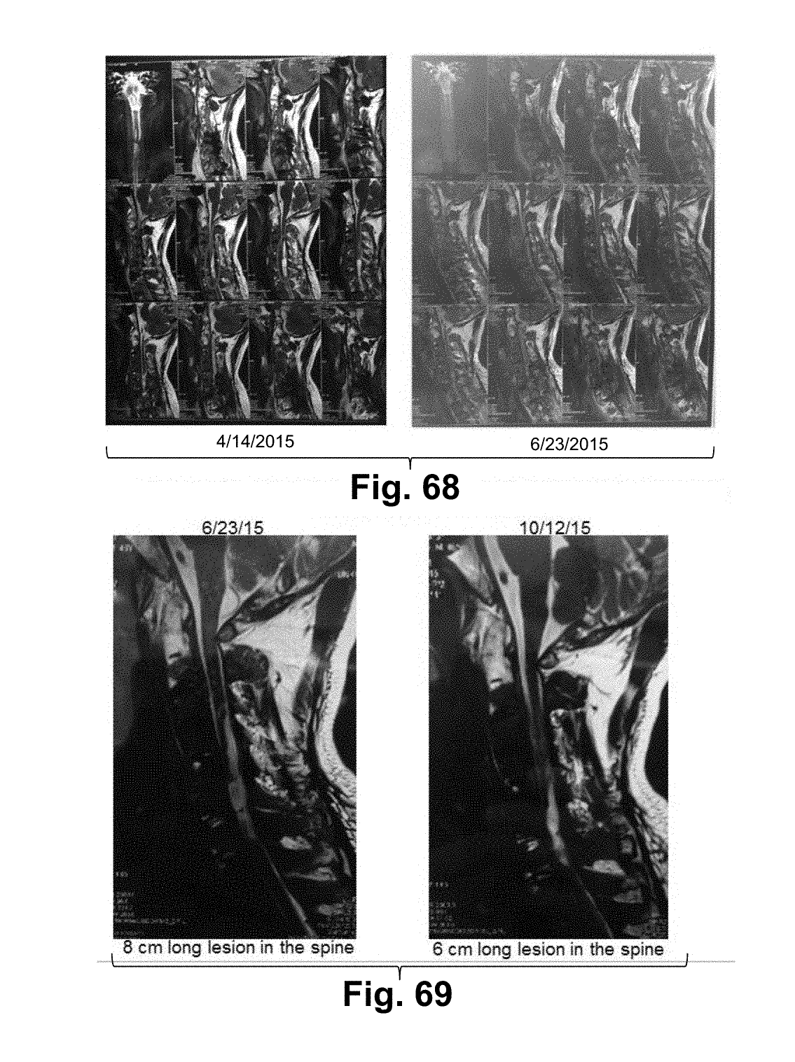

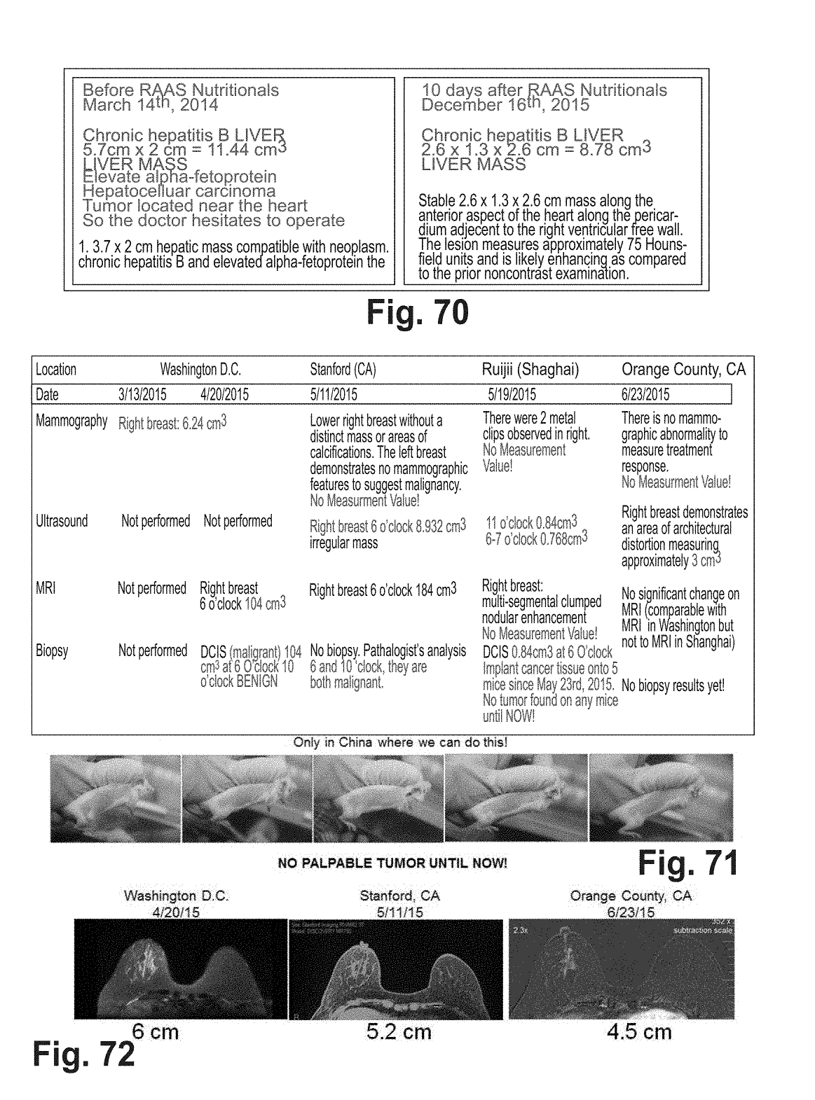

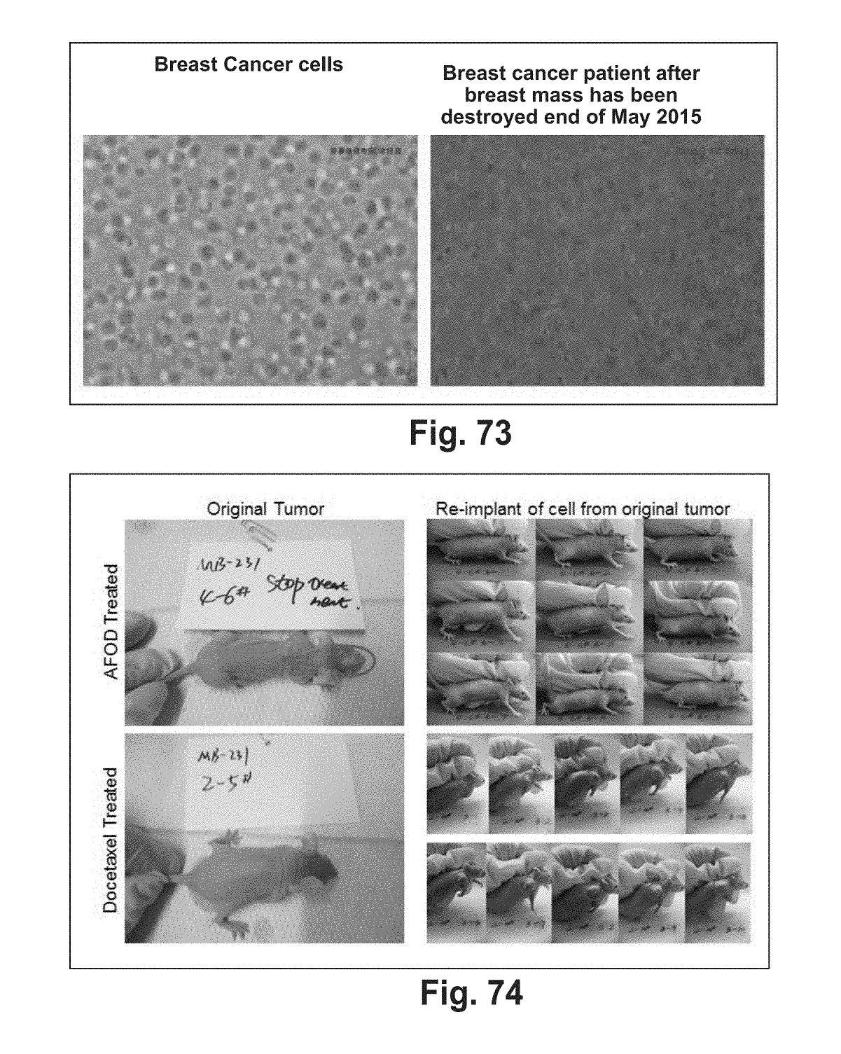

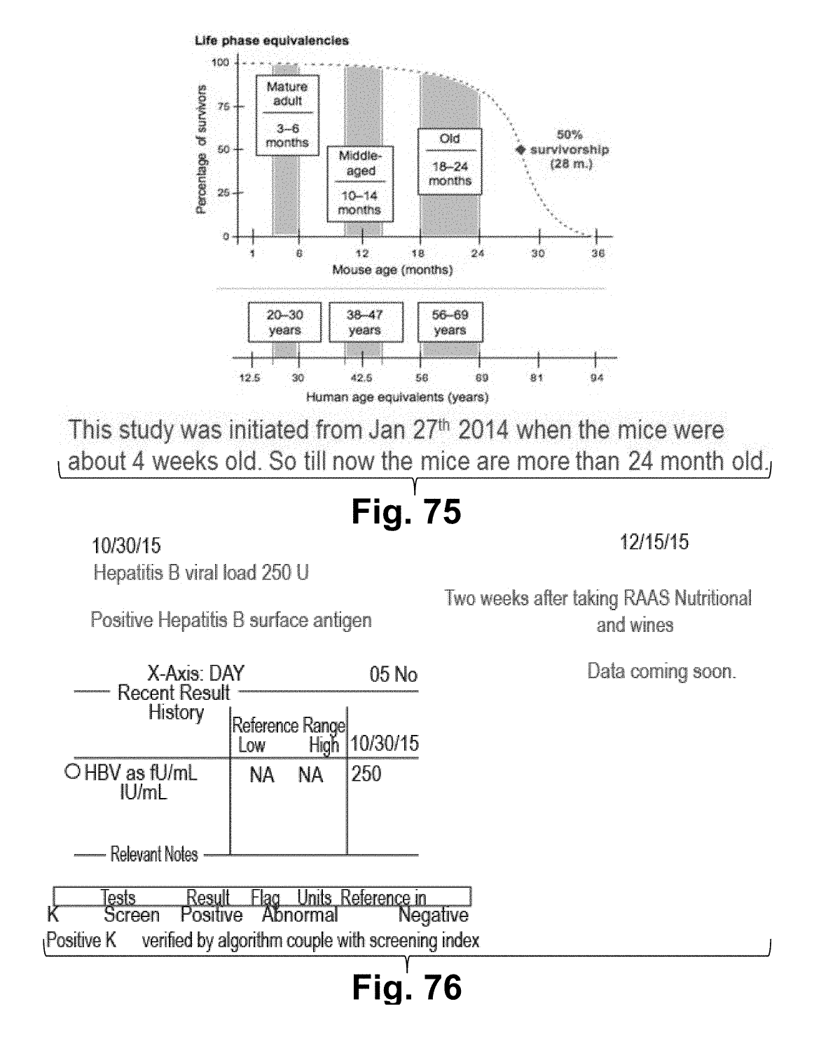

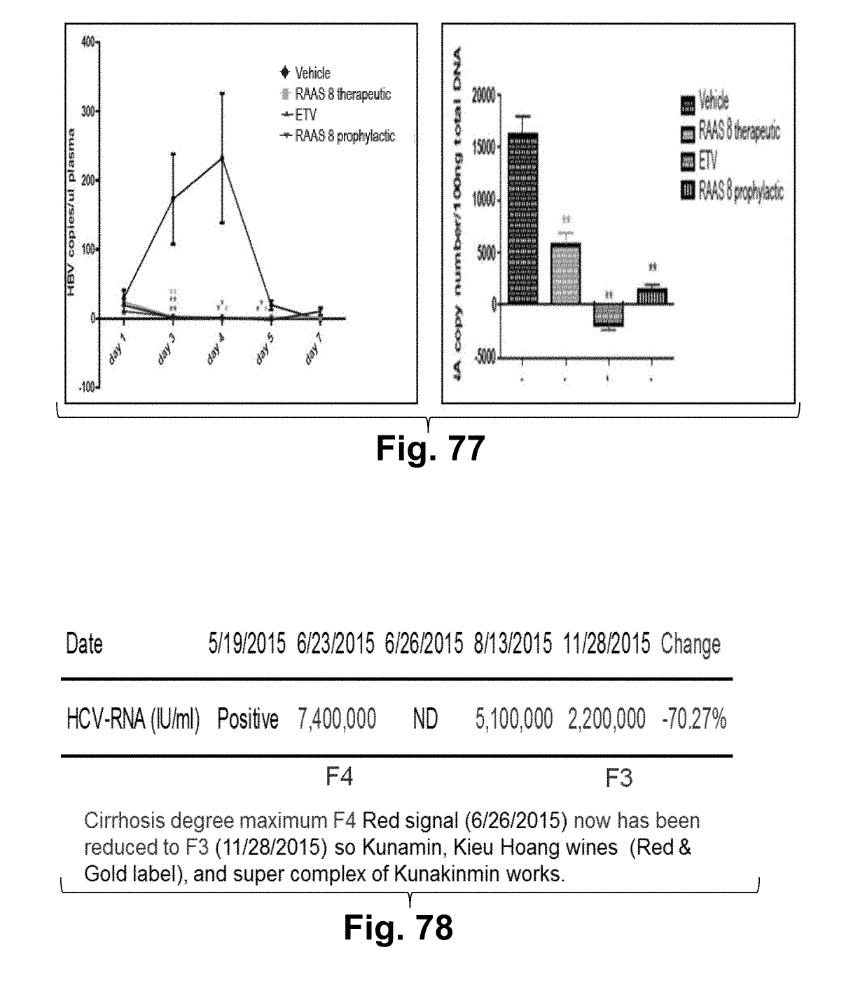

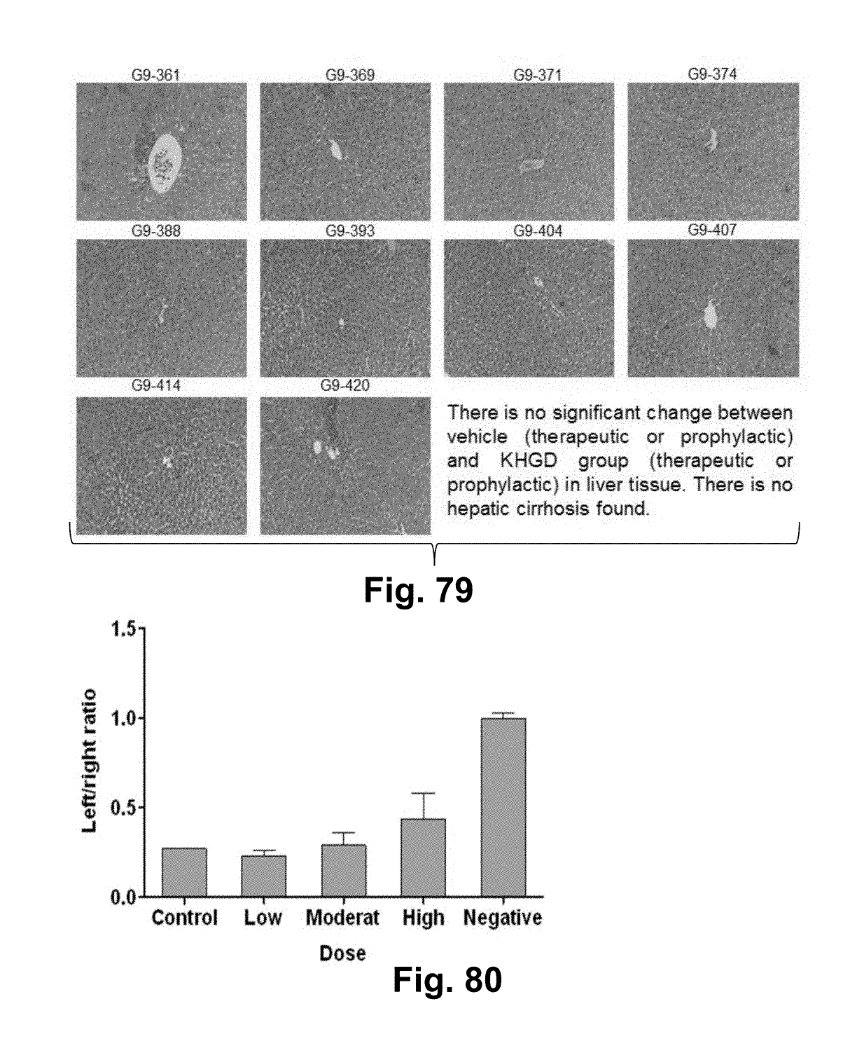

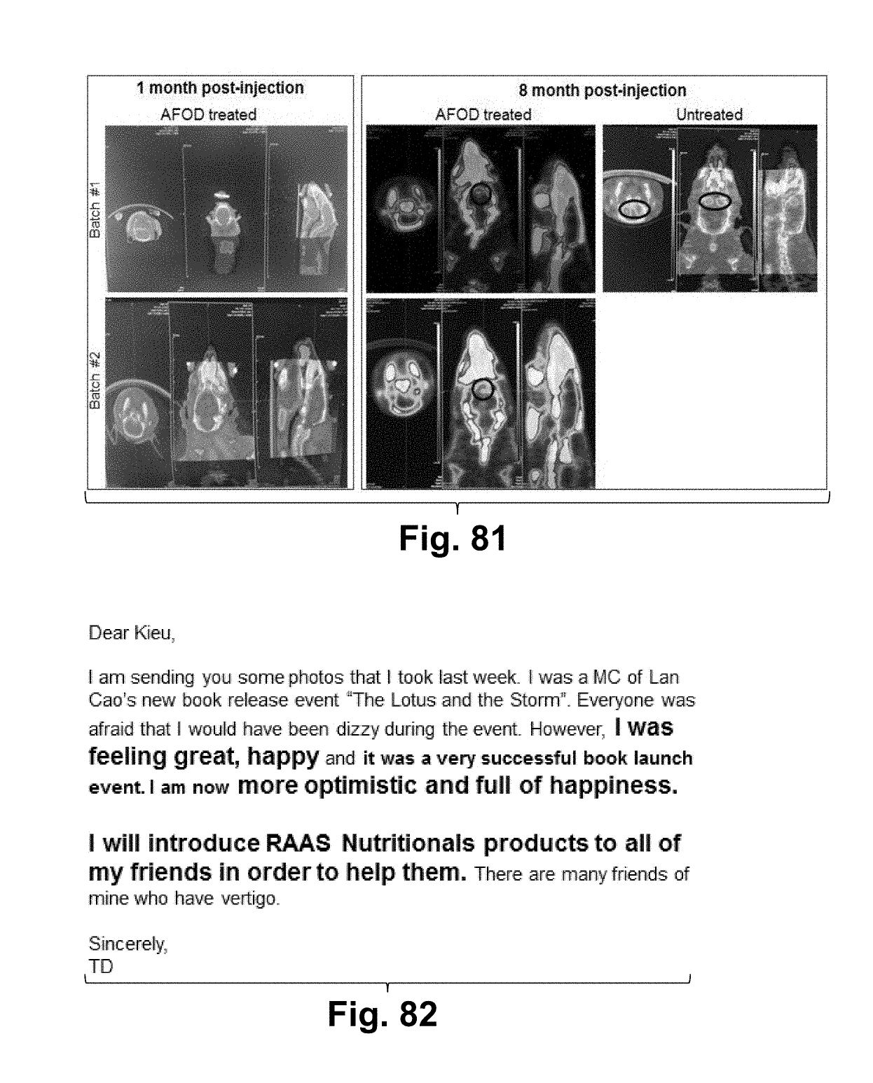

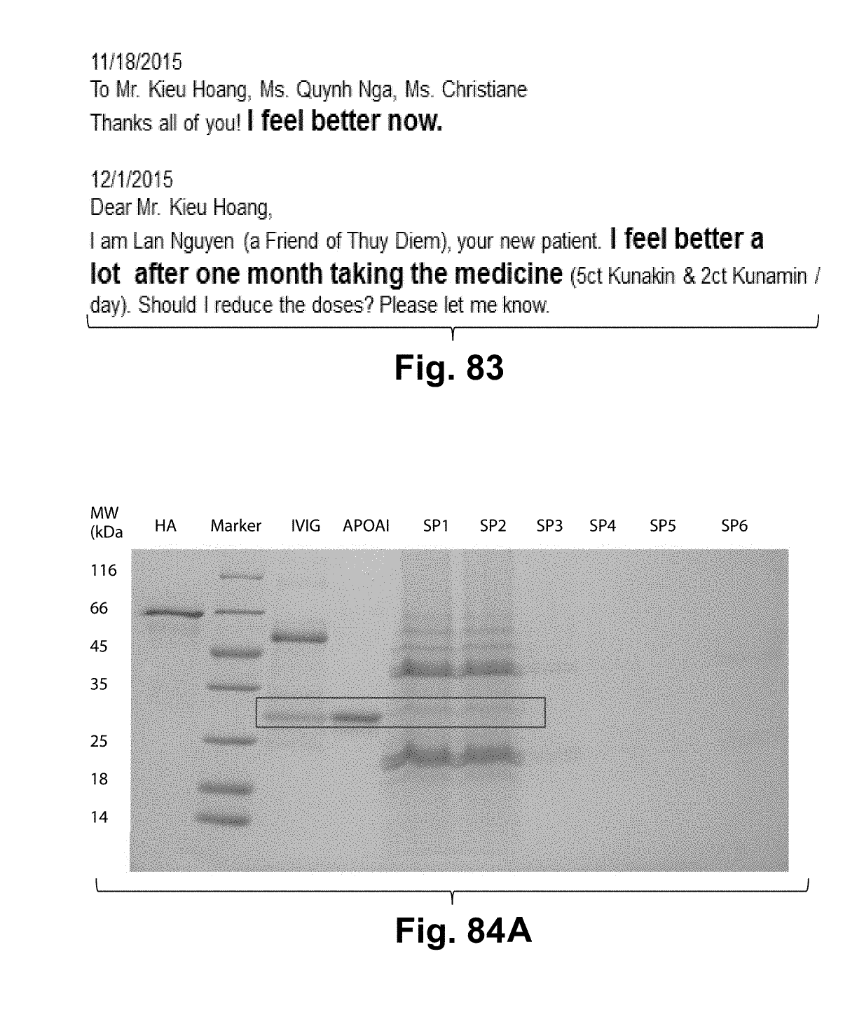

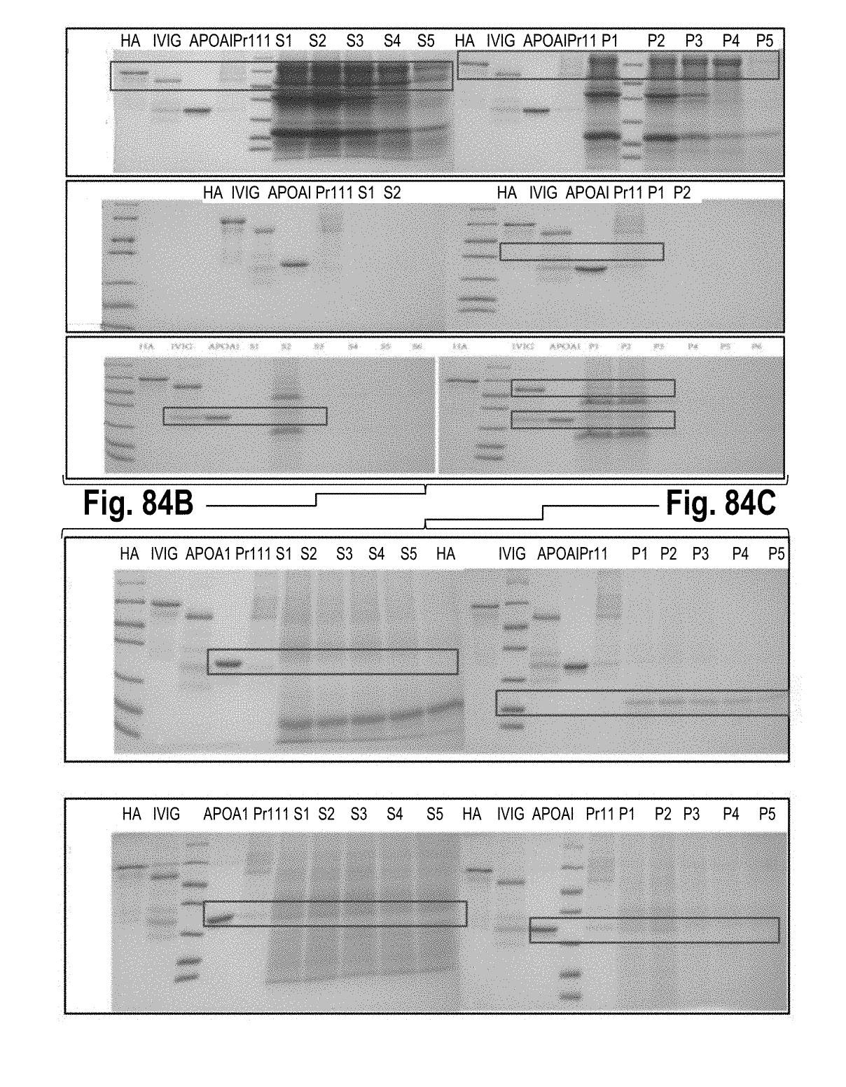

In one observational study, 51 participants signed up (FIGS. 48-51). Though a number of participants failed to return for follows-ups, qualified, complete data was collected for 22 patients with chronic kidney disease, 10 patients with diabetes, and 11 patients with cardiovascular disease. Participants reports followings after and while taking supplements for increased appetite, sleeping well, increased weight (taking more than the recommended tablets), improved energy level, and loose stools, while one participant mentioned an unpleasant smell and taste.

The majority of participants supplementing with RAAS Nutrionals as part of a daily diet noticed an improvement in overall health: 64% noticed an improvement in kidney function by an average of 9 ml/min, 65% noticed an improvement in the glycemic index by an average of 0.6 points, and participants observed an improvement on cardiovascular indicators (LDL lowered on average by 26 ml/dL; triglyceride lowered on average by 18 ml/dL; and total cholesterol lowered on average by 29 ml/dL).

With the information contained herein, various departures from precise descriptions of the present subject matter will be readily apparent to those skilled in the art to which the present subject matter pertains, without departing from the spirit and the scope of the below claims. The present subject matter is not considered limited in scope to the procedures, properties, or components defined, since the preferred embodiments and other descriptions are intended only to be illustrative of particular aspects of the presently provided subject matter. Indeed, various modifications of the described modes for carrying out the present subject matter which are obvious to those skilled in chemistry, biochemistry, or related fields are intended to be within the scope of the following claims.

I claim:

1. A food composition for human consumption comprising:
a plurality of soy ingredients,
wherein the plurality of soy ingredients comprise flesh and skin from soy, and
wherein the plurality of soy ingredients further comprise a soy construct comprising one or more of 359 soy proteins found in immature soy (KH 111) and mature soy (KH 103).

2. The food composition of claim 1, wherein the food composition is a powder or a juice.

3. The food composition of claim 2, wherein the powder is a mixed food supplement.

4. The food composition of claim 2, wherein the powder or juice comprises at least 359 proteins.

5. The food composition of claim 2, wherein the powder or juice comprises 134 proteins.

6. The food composition of claim 2, wherein the powder or juice comprises 134 proteins synthesized by 134 cells.

7. The food composition of claim 2, wherein the composition comprises Albumin from said KH 103 formulation having a molecular weight similar to Human Albumin.

8. The food composition of claim 2, wherein the composition comprises APOA1 (High Density Lipoprotein) from said KH 103 formulation having a molecular weight similar to Human APOA1 (High Density Lipoprotein).

9. The food composition of claim 2, wherein the composition comprises Alpha 1 Antitrypsin from said KH 103 formulation having a molecular weight similar to Human Alpha 1 Antitrypsin.

10. The food composition of claim 2, wherein the composition comprises Alpha 1, Alpha 2, Beta, and Gamma proteins from said KH 103 formulation having a molecular weight similar to Human Immune Globulins.

11. A method of treating certain diseases in a patient comprising administering the food composition of claim 1 to a patient in need thereof, wherein the food composition has a concentration of soy proteins found in KH 103 above 0%.

12. The method of claim 11, wherein administration of the food composition lowers triglycerides and cholesterol and increases High Density Lipoprotein (APOA1) in the patient.

13. The method of claim 11, wherein administration of the food composition cleans plaque and provides heart, brain, and artery blockage protection in the patient.

14. The method of claim 11, wherein administration of the food composition inhibits growth of various cancer cells in the patient.

15. The method of claim 11, wherein administration of the food composition suppresses inflammation in the patient due to different causes of disease.

16. A food composition for human consumption comprising a plurality of soy ingredients,
wherein the plurality of soy ingredients comprise flesh and skin from soy,
wherein the plurality of soy ingredients further comprise a soy construct comprising one or more of 359 soy proteins found in immature soy (KH 111) and mature soy (KH 103),
wherein the plurality of soy ingredients comprises a combination of at least two of 359 proteins synthesized by particular Soy KH healthy cells,
wherein the Soy KH healthy cells send signals to damaged or sick cells, thereby triggering synthesis of proteins to transform the damaged or sick cells to become healthy,
wherein the Soy KH healthy cells send signals to other undamaged cells to synthesize proteins to protect the other undamaged cells from damaged, infected, and prone to DNA and other cellular alterations, and
wherein the Soy KH healthy cells send signals to a body to produce new cells that are healthy, thereby preventing the new cells from being affected by intracellular and extracellular damaging signals.

17. A food composition for human consumption comprising a plurality of soy ingredients,
wherein the plurality of soy ingredients comprise flesh and skin from soy,
wherein the plurality of soy ingredients further comprise a soy construct comprising one or more of 359 soy proteins found in immature soy (KH 111) and mature soy (KH 103),
wherein the plurality of soy ingredients comprises a single protein from the 359 proteins synthesized by particular Soy KH healthy cells,
wherein the Soy KH healthy cells send signals to damaged or sick cells, thereby triggering synthesis of proteins to transform the damaged or sick cells to become healthy,
wherein the Soy KH healthy cells send signals to other undamaged cells to synthesize proteins to protect the other undamaged cells from being damaged, infected, and prone to DNA and other cellular alterations, and
wherein the Soy KH healthy cells send signals to a body to produce new cells that are healthy, thereby preventing the new cells from being affected by intracellular and extracellular damaging signals.

18. A method of preparing a soy construct comprising:
processing Glycine max plants immediately after harvest;
washing the Glycine max plants;
grinding the Glycine max plants and collecting raw juice;
testing pH of the raw juice;
centrifuging the raw juice and collecting a first precipitate and a first supernatant;
adding water to the first precipitate to form a water-precipitate solution, mixing the water-precipitate solution, and centrifuging the water-precipitate solution, and collecting a second precipitate and a second supernatant from the water-precipitate solution;
mixing the first supernatant, testing pH of the first supernatant, centrifuging the first supernatant, and collecting supernatant juice; and
collecting all precipitate, combining, and capsulizing the combined precipitate.

19. The method of claim 18 further comprising:
producing a final juice product by
homogenizing the supernatant juice;
sterilizing the supernatant juice;
bottling the supernatant juice; and
checking pH of the supernatant juice.

20. The method of claim 18 further comprising:
producing a final powder product by
concentrating the supernatant juice to create a concentrated juice;
spray drying the concentrated juice to produce a powder; and
packaging the powder.
